SEQUENCE LISTING (56 sequences)

SEQ ID NO: 1 - DNA, 19 bp, synthetic construct
  source 1..19: mol_type = other DNA
gaataaatgcagtatctaa

SEQ ID NO: 2 - DNA, 19 bp, synthetic construct
  source 1..19: mol_type = other DNA
ggcatggaaccaacgacaa

SEQ ID NO: 3 - DNA, 23 bp, synthetic construct
  source 1..23: mol_type = other DNA
aagcgatatattccagaaacatt

SEQ ID NO: 4 - DNA, 19 bp, synthetic construct
  source 1..19: mol_type = other DNA
gtttcgatcggatcctcaa

SEQ ID NO: 5 - DNA, 19 bp, synthetic construct
  source 1..19: mol_type = other DNA
ggtgctgtctaggcaagaa

SEQ ID NO: 6 - DNA, 19 bp, synthetic construct
  source 1..19: mol_type = other DNA
agaacattgtgaagtacaa

SEQ ID NO: 7 - DNA, 19 bp, synthetic construct
  source 1..19: mol_type = other DNA
gaatgtttaatgcaatcta

SEQ ID NO: 8 - DNA, 19 bp, synthetic construct
  source 1..19: mol_type = other DNA
ggcacactgtagtgcttaa

SEQ ID NO: 9 - DNA, 23 bp, synthetic construct
  source 1..23: mol_type = other DNA
ggcacattaatcatagataaaga

SEQ ID NO: 10 - DNA, 23 bp, synthetic construct
  source 1..23: mol_type = other DNA
atggacttagccctcaaatttca

SEQ ID NO: 11 - DNA, 19 bp, synthetic construct
  source 1..19: mol_type = other DNA
cagtatctatacagtacta

SEQ ID NO: 12 - DNA, 19 bp, synthetic construct
  source 1..19: mol_type = other DNA
gcatgttaattctcaagaa

SEQ ID NO: 13 - DNA, 19 bp, synthetic construct
  source 1..19: mol_type = other DNA
gattcaatgtgaacgatta

SEQ ID NO: 14 - DNA, 23 bp, synthetic construct
  source 1..23: mol_type = other DNA
gtgaacgattacggtctttatga

SEQ ID NO: 15 - RNA, 19 nt, synthetic construct
  source 1..19: mol_type = other RNA
gaataaatgcagtatctaa

SEQ ID NO: 16 - RNA, 19 nt, synthetic construct
  source 1..19: mol_type = other RNA
ggcatggaaccaacgacaa

SEQ ID NO: 17 - RNA, 21 nt, synthetic construct
  source 1..21: mol_type = other RNA
gcgatatattccagaaacatt

SEQ ID NO: 18 - RNA, 19 nt, synthetic construct
  source 1..19: mol_type = other RNA
gtttcgatcggatcctcaa

SEQ ID NO: 19 - RNA, 19 nt, synthetic construct
  source 1..19: mol_type = other RNA
ggtgctgtctaggcaagaa

SEQ ID NO: 20 - RNA, 19 nt, synthetic construct
  source 1..19: mol_type = other RNA
agaacattgtgaagtacaa

SEQ ID NO: 21 - RNA, 19 nt, synthetic construct
  source 1..19: mol_type = other RNA
gaatgtttaatgcaatcta

SEQ ID NO: 22 - RNA, 19 nt, synthetic construct
  source 1..19: mol_type = other RNA
ggcacactgtagtgcttaa

SEQ ID NO: 23 - RNA, 21 nt, synthetic construct
  source 1..21: mol_type = other RNA
cacattaatcatagataaaga

SEQ ID NO: 24 - RNA, 21 nt, synthetic construct
  source 1..21: mol_type = other RNA
ggacttagccctcaaatttca

SEQ ID NO: 25 - RNA, 19 nt, synthetic construct
  source 1..19: mol_type = other RNA
cagtatctatacagtacta

SEQ ID NO: 26 - RNA, 19 nt, synthetic construct
  source 1..19: mol_type = other RNA
gcatgttaattctcaagaa

SEQ ID NO: 27 - RNA, 19 nt, synthetic construct
  source 1..19: mol_type = other RNA
gattcaatgtgaacgatta

SEQ ID NO: 28 - RNA, 21 nt, synthetic construct
  source 1..21: mol_type = other RNA
gaacgattacggtctttatga

SEQ ID NO: 29 - RNA, 19 nt, synthetic construct
  source 1..19: mol_type = other RNA
ttagatactgcatttattc

SEQ ID NO: 30 - RNA, 19 nt, synthetic construct
  source 1..19: mol_type = other RNA
ttgtcgttggttccatgcc

SEQ ID NO: 31 - RNA, 21 nt, synthetic construct
  source 1..21: mol_type = other RNA
tgtttctggaatatatcgctt

SEQ ID NO: 32 - RNA, 19 nt, synthetic construct
  source 1..19: mol_type = other RNA
ttgaggatccgatcgaaac

SEQ ID NO: 33 - RNA, 19 nt, synthetic construct
  source 1..19: mol_type = other RNA
ttcttgcctagacagcacc

SEQ ID NO: 34 - RNA, 19 nt, synthetic construct
  source 1..19: mol_type = other RNA
ttgtacttcacaatgttct

SEQ ID NO: 35 - RNA, 19 nt, synthetic construct
  source 1..19: mol_type = other RNA
tagattgcattaaacattc

SEQ ID NO: 36 - RNA, 19 nt, synthetic construct
  source 1..19: mol_type = other RNA
ttaagcactacagtgtgcc

SEQ ID NO: 37 - RNA, 21 nt, synthetic construct
  source 1..21: mol_type = other RNA
tttatctatgattaatgtgcc

SEQ ID NO: 38 - RNA, 21 nt, synthetic construct
  source 1..21: mol_type = other RNA
aaatttgagggctaagtccat

SEQ ID NO: 39 - RNA, 19 nt, synthetic construct
  source 1..19: mol_type = other RNA
tagtactgtatagatactg

SEQ ID NO: 40 - RNA, 19 nt, synthetic construct
  source 1..19: mol_type = other RNA
ttcttgagaattaacatgc

SEQ ID NO: 41 - RNA, 19 nt, synthetic construct
  source 1..19: mol_type = other RNA
taatcgttcacattgaatc

SEQ ID NO: 42 - RNA, 21 nt, synthetic construct
  source 1..21: mol_type = other RNA
ataaagaccgtaatcgttcac

SEQ ID NO: 43 - RNA, 21 nt, synthetic construct
  source 1..21: mol_type = other RNA
ttctccgaacgtgtcacgttt

SEQ ID NO: 44 - RNA, 21 nt, synthetic construct
  source 1..21: mol_type = other RNA
acgtgacacgttcggagaatt

SEQ ID NO: 45 - DNA, 20 bp, synthetic construct
  source 1..20: mol_type = other DNA
tctgacttcaacagcgacac

SEQ ID NO: 46 - DNA, 20 bp, synthetic construct
  source 1..20: mol_type = other DNA
gccaaattcgttgtcatacc

SEQ ID NO: 47 - DNA, 21 bp, synthetic construct
  source 1..21: mol_type = other DNA
aacctctttgccctgtatgac

SEQ ID NO: 48 - DNA, 20 bp, synthetic construct
  source 1..20: mol_type = other DNA
ctgctcattgtcgttggttc

SEQ ID NO: 49 - RNA, 21 nt, synthetic construct
  source 1..21: mol_type = other RNA
  modified_base 20..21: mod_base = OTHER; note = thymine
  modified_base 5: mod_base = OTHER; note = pseudouridine
ggcatggaaccaacgacaatt

SEQ ID NO: 50 - RNA, 21 nt, synthetic construct
  source 1..21: mol_type = other RNA
  modified_base 20..21: mod_base = OTHER; note = thymine
  modified_base 7..8: mod_base = OTHER; note = pseudouridine
  modified_base 15: mod_base = OTHER; note = pseudouridine
  modified_base 10: mod_base = OTHER; note = pseudouridine
agaacattgtgaagtacaatt

SEQ ID NO: 51 - RNA, 21 nt, synthetic construct
  source 1..21: mol_type = other RNA
  modified_base 20..21: mod_base = OTHER; note = thymine
  modified_base 1..2: mod_base = OTHER; note = 2'-O-methyl
  modified_base 3: mod_base = OTHER; note = 2'-fluoro
  modified_base 4: mod_base = OTHER; note = 2'-O-methyl
  modified_base 5: mod_base = OTHER; note = 2'-fluoro
  modified_base 6: mod_base = OTHER; note = 2'-O-methyl
  modified_base 7: mod_base = OTHER; note = 2'-fluoro
  modified_base 8: mod_base = OTHER; note = 2'-O-methyl
  modified_base 9..11: mod_base = OTHER; note = 2'-fluoro
  modified_base 12: mod_base = OTHER; note = 2'-O-methyl
  modified_base 13: mod_base = OTHER; note = 2'-fluoro
  modified_base 14: mod_base = OTHER; note = 2'-O-methyl
  modified_base 15: mod_base = OTHER; note = 2'-fluoro
  modified_base 16: mod_base = OTHER; note = 2'-O-methyl
  modified_base 17: mod_base = OTHER; note = 2'-fluoro
  modified_base 18..19: mod_base = OTHER; note = 2'-O-methyl
agaacattgtgaagtacaatt

SEQ ID NO: 52 - RNA, 21 nt, synthetic construct
  source 1..21: mol_type = other RNA
  modified_base 20..21: mod_base = OTHER; note = thymine
  modified_base 1..2: mod_base = OTHER; note = 2'-O-methyl
  modified_base 1^2: mod_base = OTHER; note = phosphorothioate
  modified_base 3: mod_base = OTHER; note = 2'-fluoro
  modified_base 2^3: mod_base = OTHER; note = phosphorothioate
  modified_base 4: mod_base = OTHER; note = 2'-O-methyl
  modified_base 6: mod_base = OTHER; note = 2'-O-methyl
  modified_base 7: mod_base = OTHER; note = 2'-fluoro
  modified_base 8: mod_base = OTHER; note = 2'-O-methyl
  modified_base 9..11: mod_base = OTHER; note = 2'-fluoro
  modified_base 12: mod_base = OTHER; note = 2'-O-methyl
  modified_base 13: mod_base = OTHER; note = 2'-fluoro
  modified_base 14: mod_base = OTHER; note = 2'-O-methyl
  modified_base 15: mod_base = OTHER; note = 2'-fluoro
  modified_base 16: mod_base = OTHER; note = 2'-O-methyl
  modified_base 17: mod_base = OTHER; note = 2'-fluoro
  modified_base 17^18: mod_base = OTHER; note = phosphorothioate
  modified_base 18..19: mod_base = OTHER; note = 2'-O-methyl
  modified_base 18^19: mod_base = OTHER; note = phosphorothioate
  modified_base 5: mod_base = OTHER; note = 2'-fluoro
agaacattgtgaagtacaatt

SEQ ID NO: 53 - RNA, 21 nt, synthetic construct
  source 1..21: mol_type = other RNA
  modified_base 20..21: mod_base = OTHER; note = thymine
  modified_base 1..2: mod_base = OTHER; note = pseudouridine
  modified_base 4: mod_base = OTHER; note = pseudouridine
  modified_base 7..8: mod_base = OTHER; note = pseudouridine
  modified_base 11..12: mod_base = OTHER; note = pseudouridine
  modified_base 16: mod_base = OTHER; note = pseudouridine
ttgtcgttggttccatgcctt

SEQ ID NO: 54 - RNA, 21 nt, synthetic construct
  source 1..21: mol_type = other RNA
  modified_base 1..2: mod_base = OTHER; note = pseudouridine
  modified_base 4: mod_base = OTHER; note = pseudouridine
  modified_base 7..8: mod_base = OTHER; note = pseudouridine
  modified_base 14: mod_base = OTHER; note = pseudouridine
  modified_base 16..17: mod_base = OTHER; note = pseudouridine
  modified_base 19: mod_base = OTHER; note = pseudouridine
  modified_base 20..21: mod_base = OTHER; note = thymine
ttgtacttcacaatgttcttt

SEQ ID NO: 55 - RNA, 21 nt, synthetic construct
  source 1..21: mol_type = other RNA
  modified_base 20..21: mod_base = OTHER; note = thymine
  modified_base 1..3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = 2'-O-methyl
  modified_base 8: mod_base = OTHER; note = 2'-fluoro
  modified_base 9..11: mod_base = OTHER; note = 2'-O-methyl
  modified_base 12: mod_base = OTHER; note = 2'-fluoro
  modified_base 13: mod_base = OTHER; note = 2'-O-methyl
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17..19: mod_base = OTHER; note = 2'-O-methyl
ttgtacttcacaatgttcttt

SEQ ID NO: 56 - RNA, 21 nt, synthetic construct
  source 1..21: mol_type = other RNA
  modified_base 20..21: mod_base = OTHER; note = thymine
  modified_base 1^2: mod_base = OTHER; note = phosphorothioate
  modified_base 1..3: mod_base = OTHER; note = 2'-O-methyl
  modified_base 4: mod_base = OTHER; note = 2'-fluoro
  modified_base 5: mod_base = OTHER; note = 2'-O-methyl
  modified_base 6: mod_base = OTHER; note = 2'-fluoro
  modified_base 7: mod_base = OTHER; note = 2'-O-methyl
  modified_base 8: mod_base = OTHER; note = 2'-fluoro
  modified_base 9..11: mod_base = OTHER; note = 2'-O-methyl
  modified_base 12: mod_base = OTHER; note = 2'-fluoro
  modified_base 13: mod_base = OTHER; note = 2'-O-methyl
  modified_base 2^3: mod_base = OTHER; note = phosphorothioate
  modified_base 14: mod_base = OTHER; note = 2'-fluoro
  modified_base 15: mod_base = OTHER; note = 2'-O-methyl
  modified_base 16: mod_base = OTHER; note = 2'-fluoro
  modified_base 17^18: mod_base = OTHER; note = phosphorothioate
  modified_base 17..19: mod_base = OTHER; note = 2'-O-methyl
  modified_base 18^19: mod_base = OTHER; note = phosphorothioate
ttgtacttcacaatgttcttt

(19) United States
(12) Patent Application Publication
(10) Pub. No.: US 2026/0152751 A1
(43) Pub. Date: Jun. 4, 2026

(54) INTERFERING RNA FOR INHIBITING JAK1 GENE EXPRESSION AND USE THEREOF

(71) Applicant: Jenkem Technology Co., Ltd. (BEIJING), Beijing (CN)

(72) Inventors: Mai YANG, Beijing (CN); Meina Lin, Beijing (CN); Hongli Jia, Beijing (CN); Yichen Li, Beijing (CN); Xiaomeng Chen, Beijing (CN); Jun Guo, Beijing (CN); Qingbin Wang, Beijing (CN); Xuan Zhao, Beijing (CN)

(21) Appl. No.: 19/221,270

(22) Filed: May 28, 2025

(30) Foreign Application Priority Data
May 30, 2024 (CN) 202410689698.6

(51) Int. Cl. C12N 15/113

(52) U.S. Cl. CPC C12N 15/1137; C12Y 207/10002; C12N 2310/14; C12N 2310/333; C12N 2310/3341; C12N 2310/335; C12N 2310/336

(57) ABSTRACT

Provided is an interfering RNA targeting Janus kinase 1 (JAK1). The interfering RNA can reduce the expression of JAK1 and further inhibit the activation of a JAK/STAT signaling pathway, thereby achieving the purpose of treating a JAK/STAT signaling pathway dysregulation-associated disease. Further provided is a lipid nanoparticle drug containing the interfering RNA for use in treating a JAK/STAT signaling pathway dysregulation-associated disease. Further provided is an antibody-nucleic acid drug conjugate containing the interfering RNA for use in treating a JAK/STAT signaling pathway dysregulation-associated disease.

### CROSS-REFERENCE TO RELATED APPLICATIONS

This application claims priority to CN 202410689698.6 filed May 30, 2024, the entire contents of which are hereby incorporated by reference.

### REFERENCE TO SEQUENCE LISTING SUBMITTED ELECTRONICALLY

The content of the electronically submitted sequence listing (Name: 8575-0091_Sequence_Listing.xml, Size: 97,544 bytes, and Date of Creation: Jan. 29, 2026) is herein incorporated by reference in its entirety.

### TECHNICAL FIELD

The present disclosure relates to the technical field of molecular biology and biomedicine, and in particular to an interfering RNA, particularly an siRNA, for inhibiting JAK1 gene expression and use thereof.

### BACKGROUND

The JAK/STAT signaling pathway is involved in the cellular responses to various cytokines and growth factors, and was first discovered in the context of interferon (IFN) research. The JAK/STAT pathway primarily consists of three components: receptors, Janus kinases (JAKs), and signal transducers and activators of transcription (STATs). A plurality of cytokine receptors lack intrinsic kinase activity and therefore require intermediary molecules, including tyrosine kinases such as JAKs, to assist in transducing downstream signals. JAKs belong to the family of non-receptor protein tyrosine kinases and include four members: JAK1, JAK2, JAK3, and TYK2. Each JAK protein comprises seven JAK homology domains (JH). The JH1 domain located at the C-terminus is a kinase region and possesses primary catalytic activity, while the FERM (Band-4.1, ezrin, radixin, moesin) domain at the N-terminus is primarily responsible for receptor binding. Canonical activation of the JAK/STAT signaling pathway involves the following sequential mechanism: Cytokine interaction with the extracellular domain of its receptor induces receptor dimerization, which facilitates phosphorylation of conserved tyrosine residues within the kinase domains of the two JAKs constitutively associated with the receptor. Subsequently, the activated JAKs recruit and phosphorylate STATs. Phosphorylated STATs dimerize, translocate into the nucleus, and promote gene expression. To date, over 50 cytokines and growth factors have been identified to activate the JAK/STAT signaling pathway, including hormones, interferons, interleukins, and colony-stimulating factors. The downstream biological effects mediated by JAK/STAT signaling vary widely and include hematopoiesis, immune regulation, tissue repair, inflammation, apoptosis, and adipogenesis.

Dysregulation of the JAK/STAT signaling pathway is generally associated with diseases. For example, the JAK2^{V617F} mutation is commonly observed in myeloproliferative diseases. The JAK/STAT pathway also serves as a mediator of aberrantly elevated cytokines, inducing gene transcription. Therefore, treatment regimens employing JAK inhibitors (JAKi) have attracted increasing attention. In 2011, the U.S. Food and Drug Administration (FDA) approved the JAK inhibitor ruxolitinib (marketed by Novartis) for the treatment of myelofibrosis. Since then, additional JAK inhibitors have been approved for clinical use, including tofacitinib (Pfizer), baricitinib (Eli Lilly), and upadacitinib (AbbVie), for indications such as rheumatoid arthritis, ulcerative colitis, atopic dermatitis, psoriasis, and psoriatic arthritis. In addition, studies have shown that alopecia areata (AA) is associated with JAK/STAT signaling pathway and that JAK inhibitor treatment can restore hair growth in patients with severe AA (Luzhou Xing, 2014; Brittany G Craiglow, 2014). In 2022, the FDA also approved baricitinib for the treatment of adult patients with severe alopecia areata.

Currently, all marketed JAK inhibitors are small molecule drug. First-generation inhibitors exhibit low selectivity among JAK family members, and such pan-JAK inhibition may lead to multiple adverse effects. While second-generation inhibitors demonstrate improved selectivity toward specific JAK isoforms, they require high dosing frequency. Small interfering RNA (siRNA) is an RNA fragment approximately 20 nucleotides in length. In vivo, it can participate in the formation of RNA-induced silencing complexes (RISC), and target and degrade a specific mRNA through complementary base pairing, thereby suppressing protein expression. Compared to small molecule drugs, siRNA-based therapeutics can significantly reduce dosing frequency. For example, Leqvio (Novartis), indicated for treating primary hypercholesterolemia or mixed dyslipidemia in adults, requires administration only once every six months.

Therefore, the development of siRNA-based therapeutics targeting JAK1 can address specificity limitations and improve patient compliance. The present application employs siRNA to silence expression levels of JAK1 family members for treating JAK/STAT-associated diseases, such as autoimmune diseases.

### SUMMARY

In a first aspect of the present disclosure, provided is an interfering RNA targeting Janus kinase 1 (JAK1).

The interfering RNA inhibits the expression of JAK1, and the interfering RNA inhibits JAK1 gene, mRNA, and/or protein expression.

A target site sequence of the interfering RNA comprises a nucleotide sequence set forth in any one or two or more of SEQ ID NOs: 1-14. Preferably, the target site sequence of the interfering RNA is set forth in any one of the nucleotide sequences of SEQ ID NOs: 1-14.

The interfering RNA comprises one or a combination of two or more of an siRNA, a dsRNA, an shRNA, an aiRNA, or an miRNA.

In one specific embodiment of the present disclosure, the interfering RNA is an siRNA.

The interfering RNA further comprises an over-hang to increase the stability and activity of the interfering RNA.

Preferably, the interfering RNA comprises 1-10 over-hangs, further preferably, 2-4 over-hangs, e.g., 1, 2, 3, 4, 5, 6, 7, 8, 9, or 10 over-hangs.

The over-hang is located at the 3′ terminus of a sense strand and/or an antisense strand of the interfering RNA.

The over-hang is a deoxynucleoside; preferably, the over-hang may be n identical or different nucleosides (e.g., deoxythymidine (dT), deoxycytidine (dC), deoxyuridine (dU), uridine (U), guanosine (G), etc.), with n being an integer ranging from 1-10 (e.g., 1, 2, 3, 4, 5, 6, 7, 8, 9, or 10).

In one specific embodiment of the present disclosure, the terminus of the sense strand and/or the antisense strand of the interfering RNA has 2 identical over-hangs.

In one specific embodiment of the present disclosure, the terminus of the sense strand and/or the antisense strand of the interfering RNA has 2 different over-hangs.

In one specific embodiment of the present disclosure, the over-hang is dTdT, UU, or UG. Preferably, the over-hang is dTdT.

In one specific embodiment of the present disclosure, the interfering RNA comprises a sense strand and/or an antisense strand.

Preferably, the sense strand and the antisense strand are a complementary pair.

Preferably, the sense strand comprises a nucleotide sequence set forth in any one or two or more of SEQ ID NOs: 15-28. Further preferably, the sense strand is set forth in any one of the nucleotide sequences of SEQ ID NOs: 15-28.

Preferably, the antisense strand comprises a nucleotide sequence set forth in any one or two or more of SEQ ID NOs: 29-42. Further preferably, the antisense strand is set forth in any one of the nucleotide sequences of SEQ ID NOs: 29-42.

In one specific embodiment of the present disclosure, the interfering RNA comprises a nucleic acid sequence shown in Table 1.

In one specific embodiment of the present disclosure, the interfering RNA may further comprise at least one modification. The modified interfering RNA has better properties, e.g., higher stability, lower immune stimulation, etc., compared to a corresponding unmodified interfering RNA.

The modification comprises modifications on a base, a sugar ring, and/or a phosphate backbone. Preferably, the modification on a base comprises, but is not limited to, a pyrimidine modification at position 5, a purine modification at position 8, a pseudouridine modification, and/or a 5-bromouracil substitution.

Preferably, the modification on a sugar ring comprises, but is not limited to, a 2′-hydroxyl modification, a 2′-fluoro modification, a 2′-deoxy modification, a 2′-alkyl modification, a 2′-O-methyl modification, a 2′-O-methoxyethyl modification, a 2′-O-allyl modification, a 2′-C-allyl modification, and a locked nucleotide modification, including but not limited to a substitution of 2′-OH with H, OZ, Z, halo, SH, SZ, NH2, NHZ, NZ2, CN, or other groups, where Z is an alkyl group.

The alkyl group refers to a linear or branched hydrocarbon chain radical that does not contain unsaturated bonds, and is linked to the rest of the molecule via a single bond. A typical alkyl group contains 1-20 (e.g., 1, 2, 3, 4, 5, 6, 7, 8, 9, 10, 11, 12, 13, 14, 15, 16, 17, 18, 19, or 20) carbon atoms, e.g., methyl, ethyl, n-propyl, isopropyl, n-butyl, sec-butyl, isobutyl, tert-butyl, n-pentyl, isopentyl, neopentyl, tert-pentyl, n-hexyl, isohexyl, n-heptyl, isoheptyl, n-octyl, nonyl, decyl, undecyl, 1-methylundecyl, dodecyl, tridecyl, tetradecyl, pentadecyl, hexadecyl, heptadecyl, octadecyl, nonadecyl, and eicosyl.

Preferably, the modification on a phosphate backbone comprises, but is not limited to, a phosphorothioate modification and a ligand modification.

Preferably, the modification further comprises nucleotides having inosine, queuosine, xanthine, 2′-methylribose, non-natural phosphodiester bond (e.g., methylphosphonate and phosphorothioate), and/or peptide.

In one specific embodiment of the present disclosure, the modification may be methylation, fluorination, a phosphorothioate modification, pseudouridylation, etc.

Preferably, the ligand modification comprises, but is not limited to, cholesterol, biotin, a vitamin, a galactose derivative or analog, a lactose derivative or analog, N-acetylgalactosamine (GalNAc), an N-acetylgalactosamine derivative or analog, an N-acetylglucosamine derivative or analog, a mannose 6-phosphate (M6P) derivative or analog, and any combination thereof.

Preferably, the structural formula of the N-acetylgalactosamine derivative or analog has a structural formula as follows:

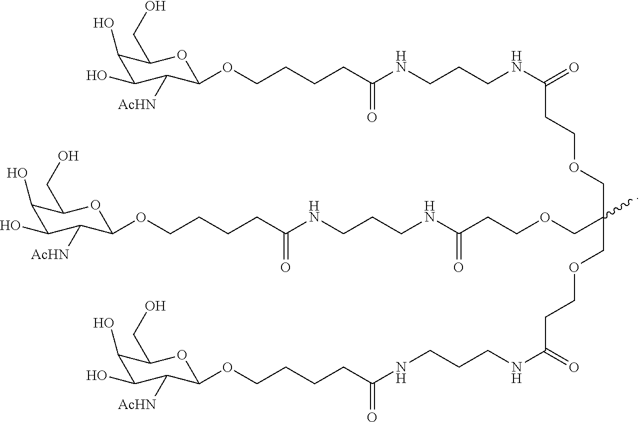 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one specific embodiment, the interfering RNA has a structural formula as follows:

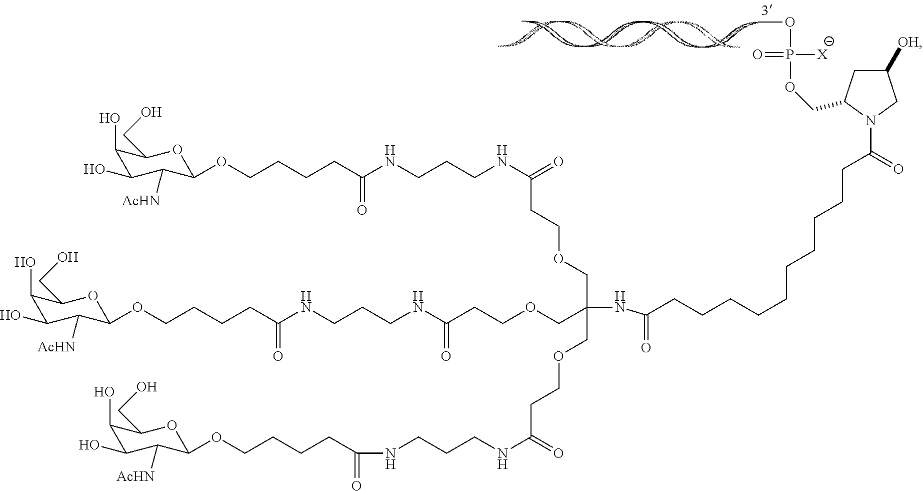

where X represents S or 0.

In one specific embodiment of the present disclosure, the sense strand of the interfering RNA is set forth in any one of the nucleotide sequences of SEQ ID NOs: 49-52;
the antisense strand of the interfering RNA is set forth in any one of the nucleotide sequences of SEQ ID NOs: 53-56.

The interfering RNA can be prepared and obtained by any method in the prior art, e.g., chemical synthesis.

In a second aspect of the present disclosure, provided is a delivery system comprising the interfering RNA described above.

Preferably, the delivery system further comprises a vector.

Preferably, the vector may be any vector suitable for delivering the interfering RNA of the present disclosure described above to a target tissue or a target cell, such as those disclosed in the prior art (e.g., Chen Zhonghua, Zhu Desheng, Li Jun, Huang Zhanqin, “Advances in non-viral vector research of siRNA”. Chinese Pharmacological Bulletin, 2015, 31 (7): 910-4; Wang Rui, Qu Bingnan, Yang Jing, “Advances in research of siRNA-carrying nano preparation”, China Pharmacy 2017, 28 (31): 4452-4455).

In one specific embodiment of the present disclosure, the vector is a viral vector; preferably, the viral vector comprises, but is not limited to, a lentivirus vector, a retrovirus vector, an adenovirus vector, an adeno-associated virus vector, a poxvirus vector, a herpesvirus vector, etc.

In one specific embodiment of the present disclosure, the vector is a non-viral vector; preferably, the non-viral vector comprises, but is not limited to, any one or a combination of two or more of a liposome, a lipid nanoparticle (LNP), a polymer, a polypeptide, an antibody, an aptamer, and N-acetylgalactosamine (GalNAc); further preferably, the non-viral vector comprises a lipid nanoparticle (LNP). The weight ratio of the interfering RNA to the non-viral vector may be any value in the range of 1:1 to 1:50, preferably any value in the range of 1:1 to 1:10 (e.g., 1:1, 1:5, 1:6, 1:7, 1:8, 1:9, 1:10, 1:15, 1:20, 1:25, 1:30, 1:35, 1:40, 1:45, or 1:50). Preferably, the lipid nanoparticle/liposome described above comprises one or a combination of two or more of a cationic lipid, a neutral lipid, a polyethylene glycol lipid, a steroidal lipid, and an anionic lipid.

Further preferably, the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]di(hexane-6,1-diyl) bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 3β-[N-(N′,N′-dimethylaminoethane)-carbamoyl]cholesterol (DC-cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA),

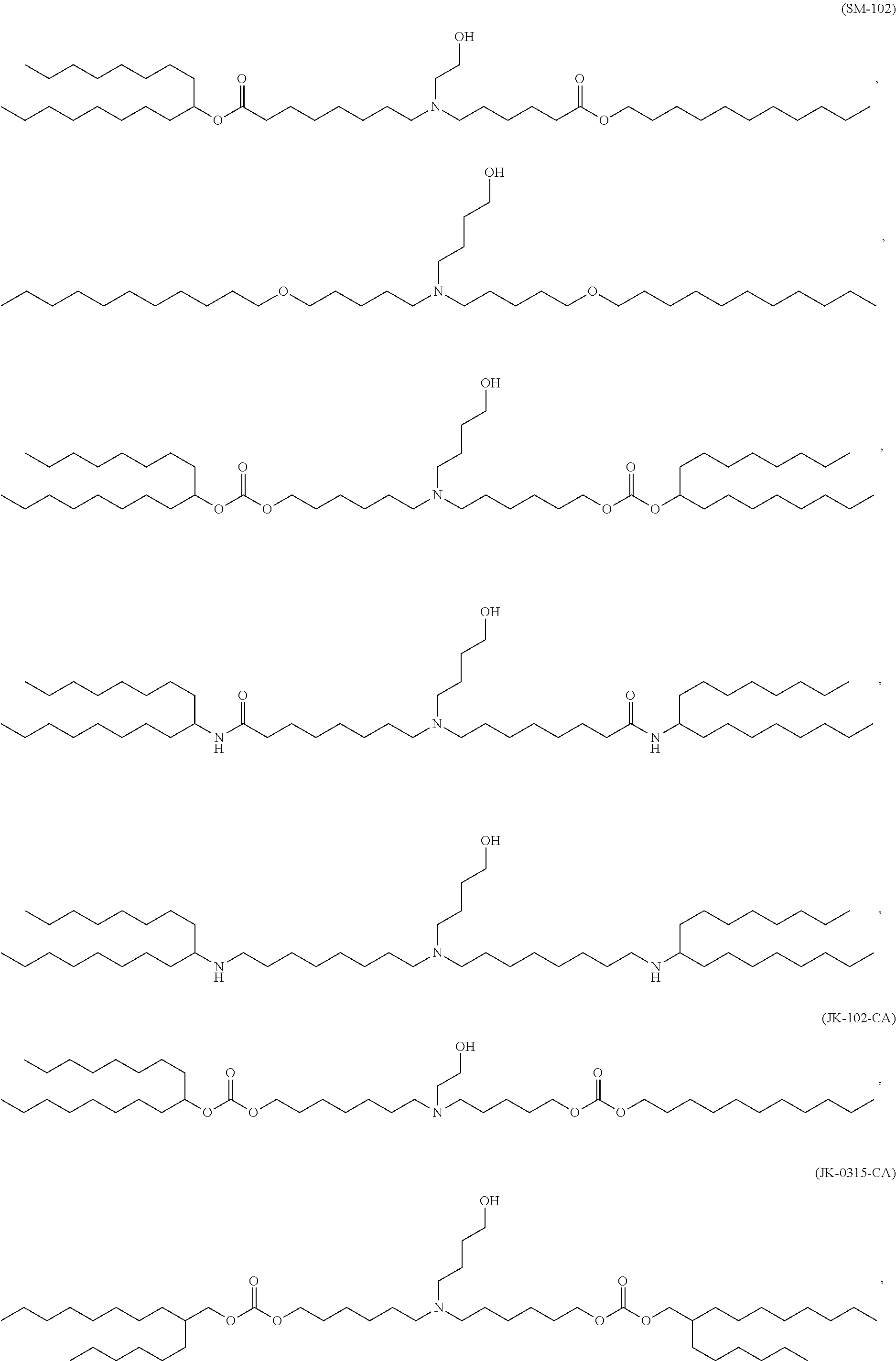

and dilinoleyl 4-(N,N-dimethylamino)butanoate (Dlin-MC3-DMA).

Preferably, the cationic lipid is a steroid-cationic lipid compound, and the compound has a structure of:

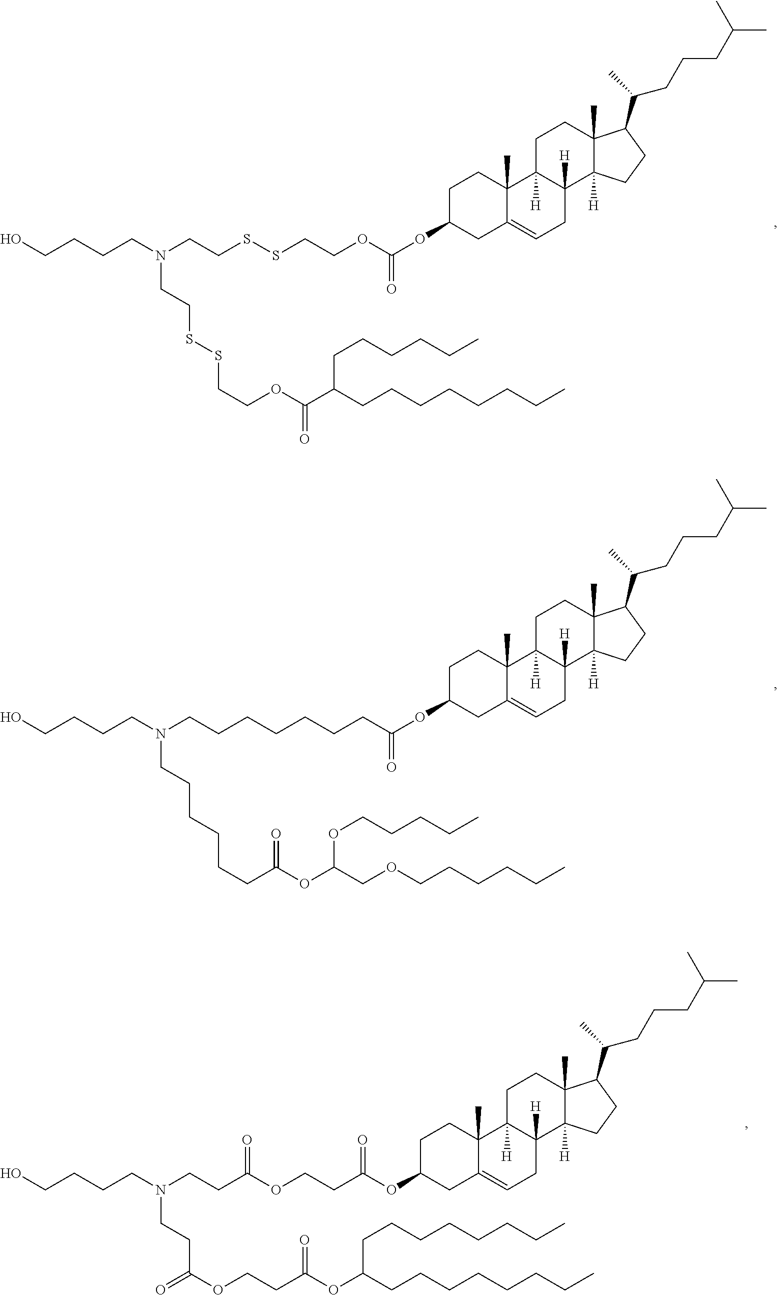

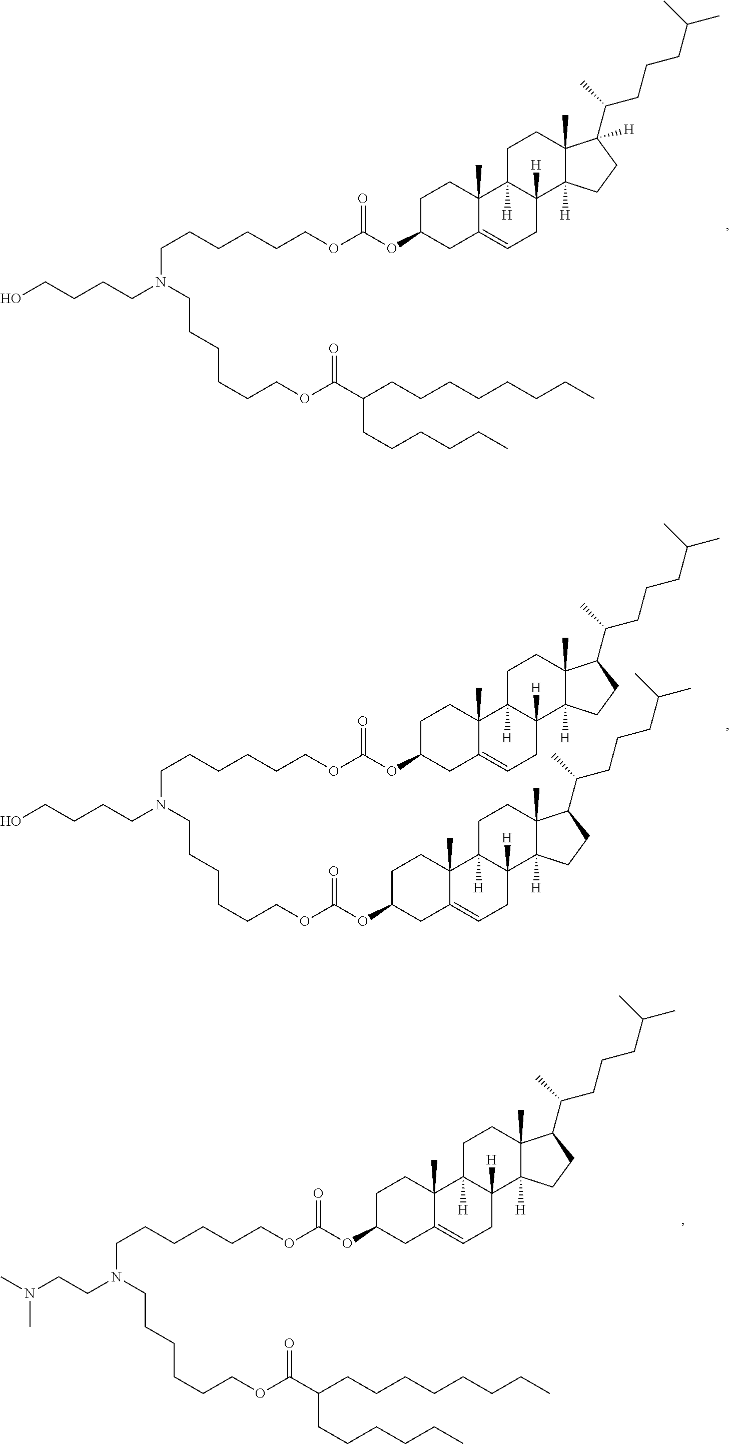

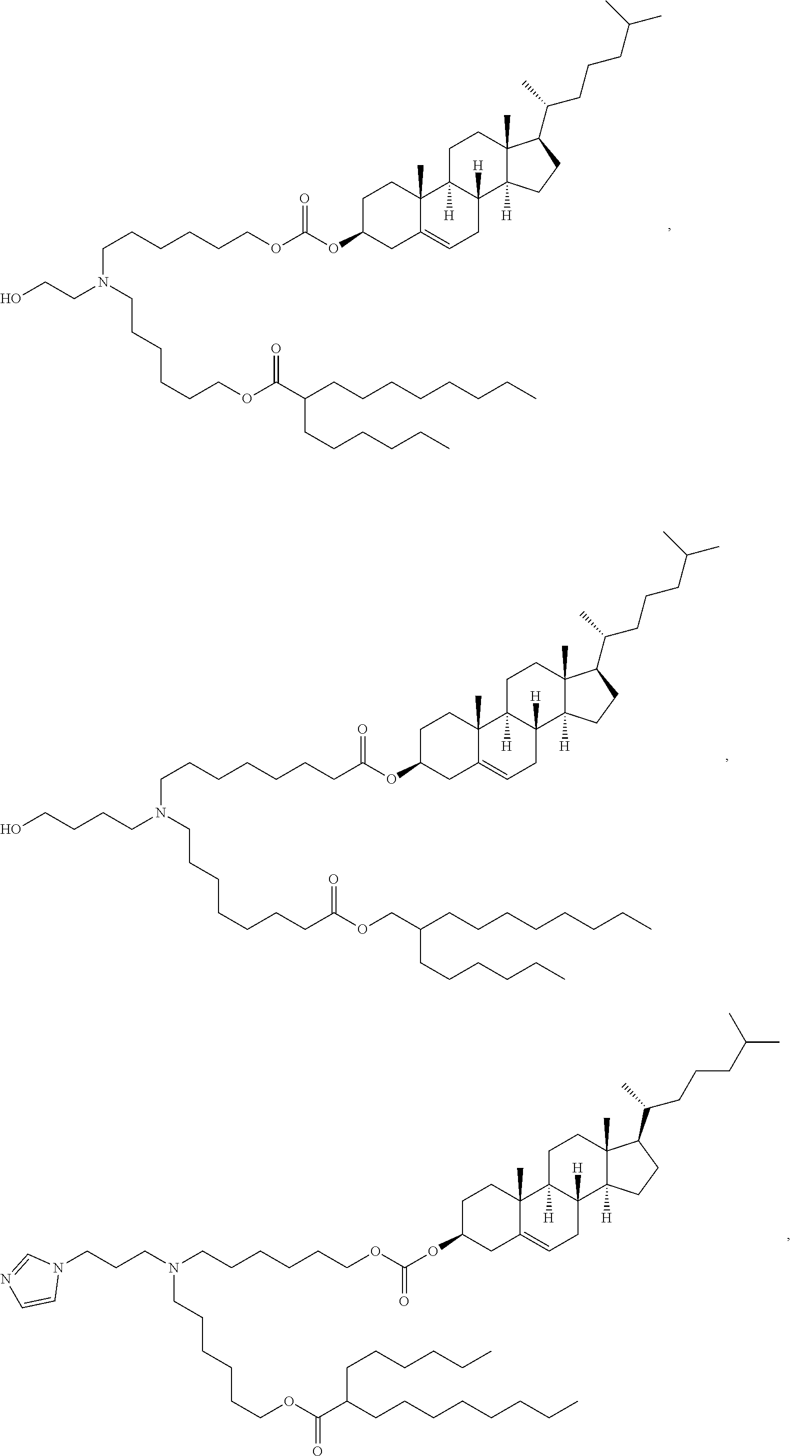

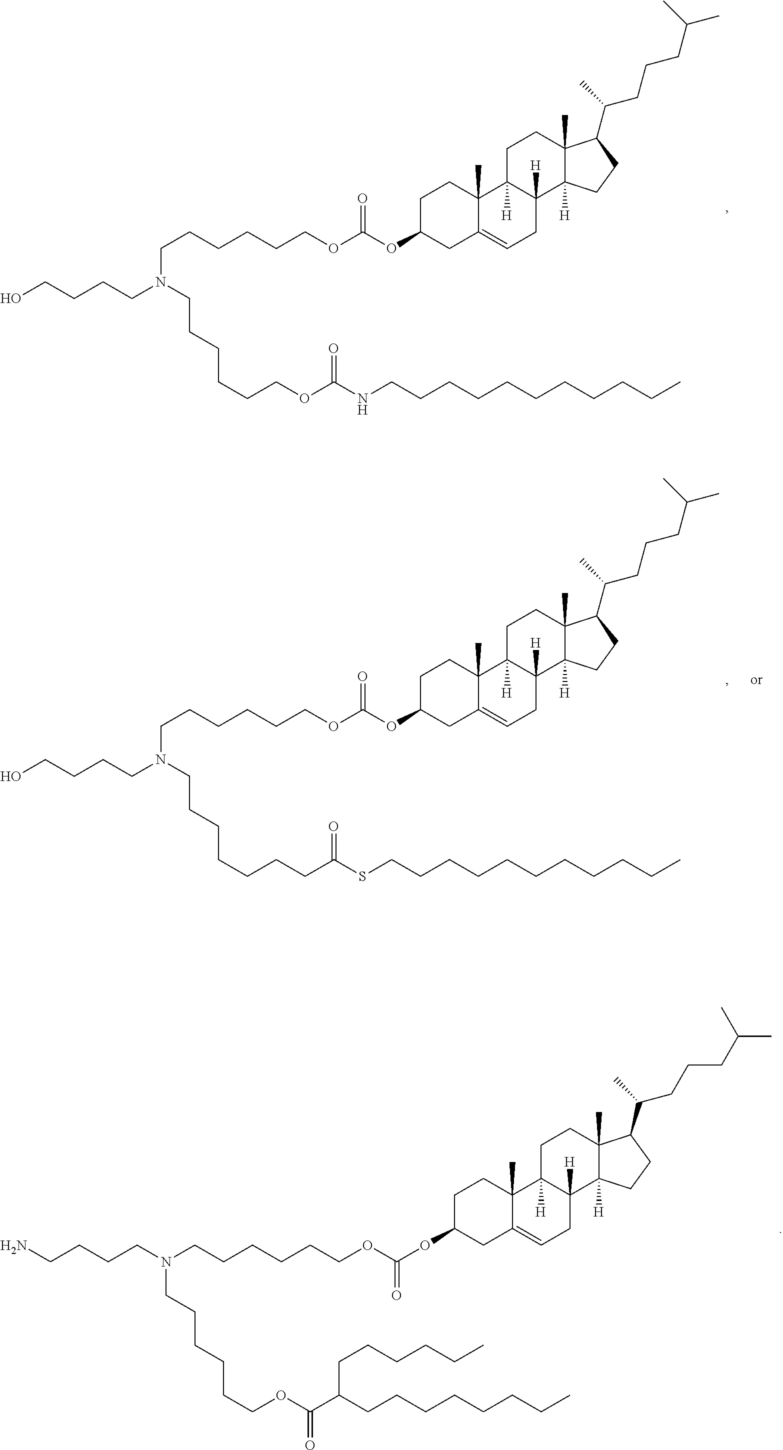

Further preferably, the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), and distearoylphosphatidylethanolamine (DSPE).

Further preferably, the polyethylene glycol lipid comprises one or a combination of two or more of 2-[(polyethylene glycol)-2000]—N,N-ditetradecylacetamide (ALC-0159), 1,2-dimyristoyl-sn-glycerol methoxypolyethylene glycol (PEG-DMG), 1,2-distearoyl-sn-glycero-3-pho sphoethanolamine-N-[amino (polyethylene glycol)](PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA),

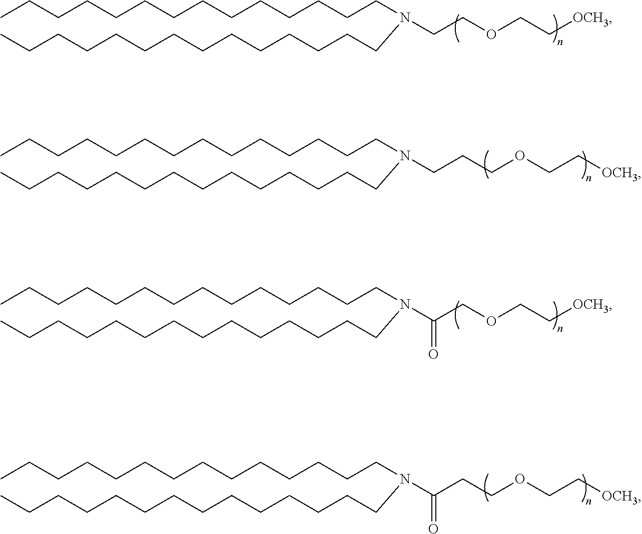

wherein n is an integer selected from 20-300, e.g., 20, 30, 50, 80, 100, 150, 200, 250, 300, etc. Preferably, the polyethylene glycol lipid is a polyethylene glycol lipid with a single molecular weight; preferably, the polyethylene glycol lipid comprises

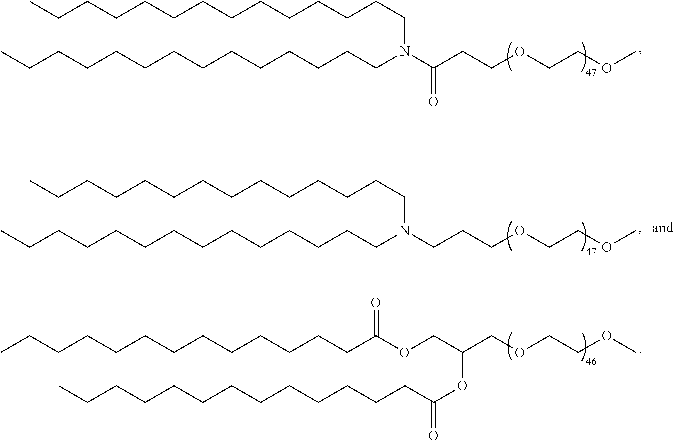

Further preferably, the anionic liposome comprises one or a combination of two or more of dioleoyl phosphatidylglycerol, dioleoyl phosphatidylethanolamine, etc.

Further preferably, the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, and lithocholic acid. Preferably, the polymer described above may be a synthetic polymer (e.g., polyethyleneimine and cyclodextrin) or a natural polymer (e.g., chitosan and atelocollagen) or a mixture thereof. Preferably, the polypeptide described above may be a cell penetrating peptide (CPP) (e.g., protamine, Tat peptide, transportan peptide, penetratin peptide, oligo-arginine peptide, etc.). Preferably, the antibody described above may be a single chain antibody (e.g., scFv-tp, scFv-9R, etc.).

The delivery system of the present application can also encapsulate various components beneficial to a human body, and directly deliver the components into cells to exert their effects, thereby achieving the desired results more quickly and effectively.

In a third aspect of the present disclosure, provided is a cell comprising the interfering RNA described above or the delivery system described above.

The expression level of JAK1 in the cell is inhibited.

The JAK1 gene, mRNA, and/or protein expression level in the cell are inhibited.

The cell may be an immortalized cell or a tumor cell.

In a fourth aspect of the present disclosure, provided is a preparation method for a cell, which comprises introducing into a cell the interfering RNA described above or the delivery system described above.

In a fifth aspect of the present disclosure, provided is a lipid nanoparticle comprising the interfering RNA described above.

Preferably, the lipid nanoparticle further comprises one or two or more of a polyethylene glycol lipid compound, a cationic lipid, a steroidal lipid, or a neutral lipid.

The polyethylene glycol lipid compound, the cationic lipid, the steroidal lipid, and the neutral lipid are as defined in the second aspect of the present application.

In one specific embodiment of the present disclosure, the lipid nanoparticle comprises the interfering RNA described above, as well as a polyethylene glycol lipid compound, a cationic lipid (excluding a steroid-cationic lipid compound), a steroidal lipid, and a neutral lipid.

Preferably, the polyethylene glycol lipid compound, the cationic lipid, the steroidal lipid, and the neutral lipid in the lipid nanoparticle are in a molar ratio of (0.5-5):(30-55):(30-55):(5-20). Preferably, the polyethylene glycol lipid compound, the cationic lipid, the steroidal lipid, and the neutral lipid are in a molar ratio of (1-5):(35-50):(40-50):(8-15).

In one specific embodiment of the present disclosure, the lipid nanoparticle comprises the interfering RNA described above, as well as a steroid-cationic lipid compound, a second lipid, and a polyethylene glycol lipid. The second lipid is selected from a neutral lipid, a zwitterionic lipid, and an anionic lipid.

Preferably, the steroid-cationic lipid, the second lipid, and the polyethylene glycol lipid in the lipid nanoparticle are in a molar ratio of 10-30:60-80:10-25, preferably 20-30:60-70:10-20.

The lipid nanoparticle of the present application can also encapsulate various components beneficial to a human body, and directly deliver the components into cells to exert their effects, thereby achieving the desired results more quickly and effectively.

The lipid nanoparticle can be prepared by using a conventional preparation method for preparing a lipid nanoparticle in the art, for example, high pressure homogenization, emulsion precipitation, ultrasonic dispersion, etc.

In a sixth aspect of the present disclosure, provided is a medicament or kit, which comprises the interfering RNA described above, the delivery system described above, the lipid nanoparticle described above, or the cell described above.

The medicament further comprises a pharmaceutically acceptable auxiliary material. Preferably, the pharmaceutically acceptable auxiliary material comprises, but is not limited to, a carrier, a diluent, a binder, a lubricant, a wetting agent, etc.

Preferably, an administration mode of the medicament includes, but is not limited to, oral administration, intestinal administration, subcutaneous injection, intramuscular injection, intravenous injection, nasal administration, transdermal administration, subconjunctival administration, intraocular administration, orbital administration, retrobulbar administration, retinal administration, choroidal administration, intrathecal injection, etc.

Preferably, the dosage form of the medicament includes, but is not limited to, a tablet, a capsule, a pill, an injection, an inhalant, a troche, a suppository, an emulsion, a microemulsion, a submicroemulsion, a nanoparticle, a gel, a powder, a suspoemulsion, a cream, a jelly, a spray, etc. The various dosage forms of the medicament can be prepared according to conventional production methods in the field of pharmaceuticals.

Preferably, the mass content of the interfering RNA described above, the delivery system described above, or the cell described above in the medicament may be 1%-100%, for example, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, or 100%.

In a seventh aspect of the present disclosure, provided is an antibody-nucleic acid drug conjugate, which comprises the interfering RNA described above or the delivery system described above or the lipid nanoparticle described above.

The antibody-nucleic acid drug conjugate comprises one or more interfering RNAs.

In the antibody-nucleic acid drug conjugate, the antibody and the nucleic acid may be directly linked or linked via a linking group or a linker peptide.

The antibody-nucleic acid drug conjugate has a general formula (I):

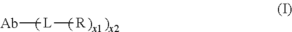

wherein R is the interfering RNA described above;
x1 is an integer ranging from 1-144; preferably, x1 is an integer ranging from 1-9; more preferably,
x1 is an integer ranging from 1-3;
x2 is an integer ranging from 1-8; preferably, x2 is an integer ranging from 1-2;
Ab is an antibody;
L is a linking unit connecting Ab and R.

Preferably, the moiety L has a structure of general formula (II):

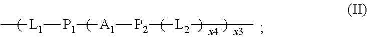

wherein
x3 in formula II is an integer selected from 1-12, preferably 1-3;
x4 in formula II is an integer selected from 1-12, preferably 1-3;
P₁ and P₂ are identical or different polyethylene glycol residues;
L₁ is a linking unit connecting Ab and P₁;
L₂ is a linking unit connecting P₂ and R;
A₁ is a linking unit connecting P₁ and P₂;
preferably, P₁ and P₂ are independently selected from linear, Y-shaped, and multi-branched polyethylene glycol residues;
preferably, when P₁ and P₂ are polyethylene glycol with a single molecular weight, the molecular weight is 88-4400 Da, and more preferably, the molecular weight of P₁ and P₂ is 176-1056 Da; preferably, when P₁ and P₂ are polyethylene glycol with a non-single molecular weight, the molecular weight is 1000 Da-40 kDa; and more preferably, the molecular weight of P₁ and P₂ is 2000 Da-10 kDa.

Preferably, L₁ is a linking group selected from one or a combination of two or more of linear or branched C₁₋₁₂ alkylene, C₆₋₁₂ arylene, C₃₋₁₂ cycloalkylene, —S—,

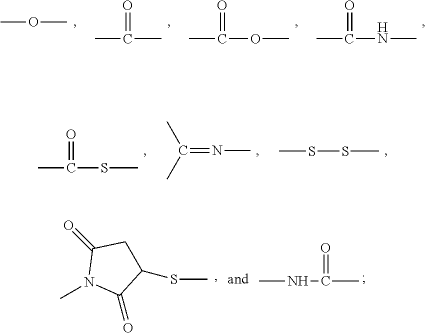

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl or substituted or unsubstituted heterocyclylalkyl,

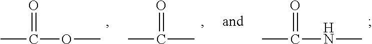

preferably, L₂ is a linking group selected from one or a combination of two or more of linear or branched C₁₋₁₂ alkylene, C₆₋₁₂ arylene, C₃₋₁₂ cycloalkylene, —S—,

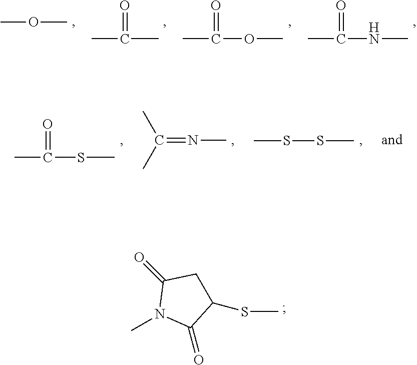

an optional H atom on the linear or branched C₁₋₁₂ chain alkylene, C₆₋₁₂ arylene, or C₃₋₁₂ cycloalkylene is substituted with one or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl or substituted or unsubstituted heterocyclylalkyl,

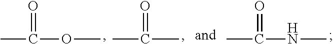

preferably, L₁ is an amide bond, a hydrazone bond, or a thiol-maleimide bond;
more preferably, L₁ is an amide bond;
preferably, L₂ is a disulfide bond or a thiol-maleimide bond; and
more preferably, L₂ is a disulfide bond.

Preferably, A₁ is a linking group connecting P₁ and P₂, and is selected from one or a combination of two or more of linear or branched C₁₋₁₂ alkylene, C₆₋₁₂ arylene, C₃₋₁₂ cycloalkylene, —S—,

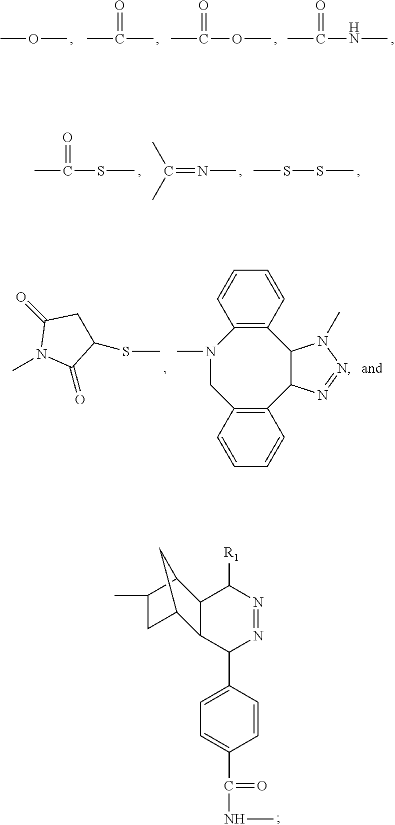

an optional H atom on the linear or branched C₁₋₁₂ chain alkylene, C₆₋₁₂ arylene, or C₃₋₁₂ cycloalkylene is substituted with one or a combination of two or more of —H, —F, —Cl, —Br, —I, —O—, —S—, —SO₂, —NO₂, C₁₋₁₂ chain alkyl, C₃₋₁₂ cycloalkyl, C₆₋₁₂ aralkyl, substituted or unsubstituted heterocyclyl or substituted or unsubstituted heterocyclylalkyl,

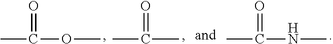

Further preferably, the antibody-nucleic acid drug conjugate has a general formula (IV):

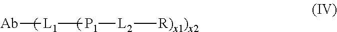

wherein L₁ is a linking group selected from one or a combination of two or more of linear or branched C₁₋₁₂ alkylene, C₆₋₁₂ arylene, C₃₋₁₂ cycloalkylene, —S—,

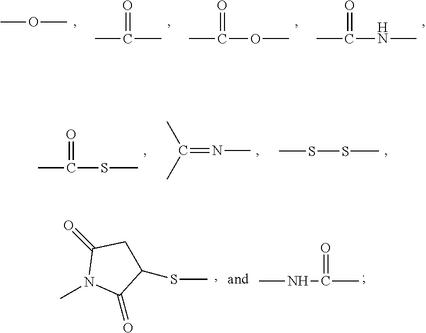

preferably, P₁ are identical or different polyethylene glycol residues; preferably, when P₁ is polyethylene glycol with a single molecular weight, the molecular weight is 88-4400 Da; more preferably, the molecular weight of P₁ is 176-1056 Da;
preferably, when P₁ is a polyethylene glycol with a non-single molecular weight, the molecular weight is 1000 Da-40 kDa; more preferably, the molecular weight of P₁ is 2000 Da-10 kDa.

L₂ is a linking group selected from one or a combination of two or more of linear or branched C₁₋₁₂ alkylene, C₆₋₁₂ arylene, C₃₋₁₂ cycloalkylene, —S—,

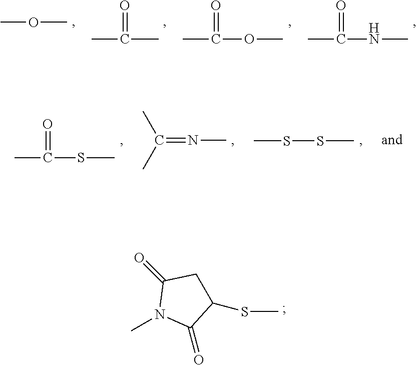

x1 is an integer ranging from 1-144; preferably, x1 is an integer ranging from 1-9; more preferably,
x1 is an integer ranging from 1-3;
x2 is an integer ranging from 1-8; preferably, x2 is an integer ranging from 1-2.

In one specific embodiment of the present disclosure, the antibody-nucleic acid drug conjugate has the following structure:

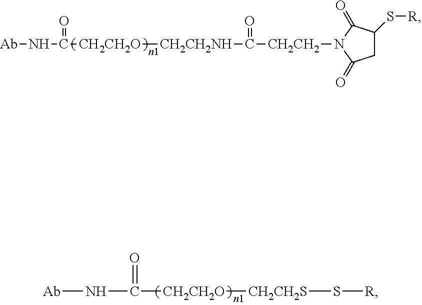

wherein n₁ is an integer selected from 4-100, preferably 4-24; n1 may be a constant value or an average value.

In one specific embodiment of the present disclosure, the antibody-nucleic acid drug conjugate has the following structure:

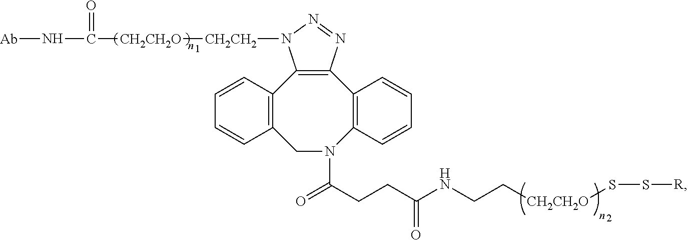

wherein n₁ and n₂ are independently selected from integers ranging from 4-100, and preferably 4-24. n1 and n2 may be constant values or average values.

Preferably, Ab is selected from a monoclonal antibody, a polyclonal antibody, an antibody fragment, and an antibody fusion fragment.

The antibody may be a single domain antibody or a single chain antibody.

Further preferably, Ab is a monoclonal antibody; more preferably, the monoclonal antibody is reactive to an antigen or an epitope thereof associated with a cancer, a malignant cell, an infectious organism, or an autoimmune disease.

In one specific embodiment of the present disclosure, the Ab is selected from an anti-HER2 antibody, an anti-EGFR antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFR VIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, an anti-Muc antibody, an anti-CD3 antibody, an anti-CD28 antibody, an anti-CD38 antibody, an anti-CD19 antibody, an anti-PD-L1 antibody, and an anti-4-1BB antibody. In an eighth aspect of the present disclosure, provided is use of the interfering RNA described above, the delivery system described above, the cell described above, the medicament or the kit described above, the lipid nanoparticle described above, or the antibody-nucleic acid drug conjugate described above, which comprises
A) use in preparing a medicament for treating and/or preventing a JAK/STAT signaling pathway dysregulation-associated disease;
B) use in inhibiting JAK1 expression; and
C) use in treating and/or preventing a JAK/STAT signaling pathway dysregulation-associated disease.

Preferably, the JAK/STAT signaling pathway dysregulation-associated disease (diseases associated with the increase of the expression of JAK1 or the excessive proliferation of cells) comprises, but is not limited to, an autoimmune diseases or a tumor.

Further preferably, the autoimmune disease comprises, but is not limited to, rheumatoid arthritis, atopic dermatitis, vitiligo, psoriasis, psoriatic arthritis, or alopecia areata.

Further preferably, the tumor comprises a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, blood cancer, mesothelioma, myeloma (including myeloproliferative neoplasm (MPN)), a myelodysplastic syndrome, a myeloproliferative disease, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

The myelodysplastic syndrome (MDS) includes, but is not limited to, refractory anemia, refractory anemia with ring sideroblasts, refractory anemia with excess blasts, refractory anemia with excess blasts in transformation, refractory cytopenia with multilineage dysplasia, myelodysplastic syndrome with 5q-syndrome, and therapy-related myelodysplastic syndrome.

The myeloproliferative disease (MPD) includes, but is not limited to, chronic myeloid leukemia (CML), polycythemia vera (PV), essential thrombocythemia (ET), primary or idiopathic myelofibrosis (PMF), chronic neutrophilic leukemia, chronic eosinophilic leukemia, chronic myelomonocytic leukemia, juvenile myelomonocytic leukemia, eosinophilia, and systemic mastocytosis.

In a ninth aspect of the present disclosure, provided is a method for inhibiting JAK1 expression, which comprises adding the interfering RNA described above, the delivery system described above, the cell described above, the lipid nanoparticle described above, the medicament or the kit described above, or the antibody-nucleic acid drug conjugate described above.

Preferably, the method inhibits JAK1 expression in a cell, and the cell is an immortalized cell or a tumor cell.

Preferably, the method comprises delivering into a cell the interfering RNA described above. The delivery is performed using the delivery system described above.

In a tenth aspect of the present disclosure, provided is a method for treating a JAK/STAT signaling pathway dysregulation-associated disease, which comprises administering to a subject a therapeutically effective amount of the interfering RNA described above, the delivery system described above, the lipid nanoparticle described above, the cell described above, the medicament or the kit described above, or the antibody-nucleic acid drug conjugate described above. Preferably, the JAK/STAT signaling pathway dysregulation-associated disease comprises, but is not limited to, an autoimmune disease or a tumor.

Further preferably, the autoimmune disease comprises, but is not limited to, rheumatoid arthritis, atopic dermatitis, vitiligo, psoriasis, psoriatic arthritis, or alopecia areata.

Further preferably, the tumor comprises a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, blood cancer, mesothelioma, myeloma (including myeloproliferative neoplasm (MPN)), a myelodysplastic syndrome, a myeloproliferative disease, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

The myelodysplastic syndrome (MDS) includes, but is not limited to, refractory anemia, refractory anemia with ring sideroblasts, refractory anemia with excess blasts, refractory anemia with excess blasts in transformation, refractory cytopenia with multilineage dysplasia, myelodysplastic syndrome with 5q-syndrome, and therapy-related myelodysplastic syndrome.

The myeloproliferative disease (MPD) includes, but is not limited to, chronic myeloid leukemia (CML), polycythemia vera (PV), essential thrombocythemia (ET), primary or idiopathic myelofibrosis (PMF), chronic neutrophilic leukemia, chronic eosinophilic leukemia, chronic myelomonocytic leukemia, juvenile myelomonocytic leukemia, eosinophilia, and systemic mastocytosis.

Currently marketed JAK inhibitors are all small molecule drugs that can suppress the kinase activity of JAKs; however, they require frequent administration and are associated with significant adverse effects. Compared with small molecule drugs, siRNA-based therapeutics offer the advantages of high target specificity and reduced dosing frequency. The JAK1 siRNA sequences screened in the present application can be used in treating a JAK/STAT signaling pathway dysregulation-associated disease, such as an autoimmune disease, and possess strong development potential. Experimental results demonstrate that siJAK1 can significantly reduce the mRNA expression level of JAK1 in cells. Therefore, it is expected to be used in the treatment of diseases associated with elevated JAK1 expression, such as an autoimmune disease. By inhibiting Janus kinase expression and modulating the JAK/STAT signaling pathway, the objectives of slowing disease progression and improving disease outcomes are achieved.

The term “interfering RNA” as described herein includes a single-stranded RNA (e.g., mature miRNA, ssRNA oligonucleotide, and ssDNA oligonucleotide) or a double-stranded RNA (e.g., siRNA, dsRNA, shRNA, aiRNA, or pre-miRNA) which is capable of reducing or inhibiting expression of a target gene or sequence (e.g., by mediating degradation and/or inhibiting translation of mRNA complementary to the interfering RNA in sequence) when the interfering RNA and the target gene or sequence are in the same cell.

The siRNA is a small interfering RNA, and each strand of the siRNA molecule comprises nucleotides having a length of about 15 to about 60 nucleotides (e.g., nucleotides having a length of about 15-60, 15-50, 15-40, 15-30, 15-25, or 19-25 nucleotides, or nucleotides having a length of 15, 16, 17, 18, 19, 20, 21, 22, 23, 24, or 25 nucleotides). In one specific embodiment, the siRNA may be chemically synthesized. The siRNA molecule of the present disclosure can silence the expression of a target sequence in vitro and/or in vivo. In some embodiments, the siRNA may not contain modified nucleotides; in other embodiments, the siRNA comprises at least one modified nucleotide. For example, the siRNA comprises one, two, three, four, five, six, seven, eight, nine, ten, or more modified nucleotides in the double-stranded region. The dsRNA or pre-RNA molecules include any precursor molecule that is processed in vivo by an endonuclease to produce an active siRNA. shRNA is a small hairpin RNA or short hairpin RNA comprising short RNA sequences producing tight hairpin turns that can be used to silence gene expression via RNA interference. The shRNA hairpin structure can be lysed by a cellular machinery to siRNA. The miRNA (microRNA) is a single-stranded RNA molecule that regulates gene expression and has a length of about 21-23 nucleotides.

“Inhibiting the expression of a target gene” as described herein refers to the ability of the interfering RNA (e.g., siRNA) of the present disclosure to silence, reduce, or inhibit the expression of a target gene (e.g., JAK gene).

The term “comprise”, “comprising”, or “include” as described herein is an open-ended description that includes the specified ingredients or steps as described, as well as other specified ingredients or steps that do not substantially affect the technical effect. When the term is used to describe a sequence of a protein or nucleic acid, the protein or nucleic acid may consist of the sequence, or may have additional amino acids or nucleotides at one or two ends of the protein or nucleic acid, while retaining the same or similar activity as the original sequence.

The term “tumor” as described herein may be any adverse cell proliferation (or any disease that manifests itself as adverse cell proliferation) or neoplasm, or increased predisposition or risk for adverse cell proliferation, neoplasm, or a tumor. The tumor may be benign or malignant, and may also be primary or secondary (metastatic). The neoplasm may be any abnormal growth or proliferation of cells and may be located in any tissue. Examples of the tissue include adrenal gland, adrenal medulla, anus, appendix, bladder, blood, bone, bone marrow, brain, mammary gland, cecum, central nervous system (including or excluding brain), cerebellum, cervix, colon, duodenum, endometrium, epithelial cells (e.g., renal epithelial cells), gallbladder, esophagus, glial cells, heart, ileum, jejunum, kidney, lacrimal gland, larynx, liver, lung, lymph, lymph nodes, lymphoblasts, maxilla, mediastinum, mesenterium, myometrium, nasopharynx, omentum, oral cavity, ovary, pancreas, parotid gland, peripheral nervous system, peritoneum, pleura, prostate, salivary glands, sigmoid colon, skin, small intestine, soft tissue, spleen, stomach, testis, thymus, thyroid, tongue, tonsils, trachea, uterus, vulva, and white blood cells. Further preferably, the tumor comprises a gastrointestinal tumor (oral cancer, tongue cancer, esophageal cancer, gastric cancer, liver cancer, pancreatic cancer, colorectal cancer, etc.), a nervous system tumor (glioma (e.g., neuroglioma), neuroepithelioma, schwannoma, astrocytoma, neurofibroma (e.g., neurofibrosarcoma), ependymoma, medulloblastoma, meningioma, brain metastasis, etc.), a respiratory tract tumor (nasopharyngeal carcinoma, laryngeal carcinoma, bronchial carcinoma, lung cancer, etc.), a urinary system tumor (e.g., prostate cancer, renal cell carcinoma, bladder cancer, etc.), a reproductive system tumor (breast cancer, cervical cancer, ovarian cancer, placental choriocarcinoma, etc.), a hematological and lymphatic system tumor (multiple myeloma, blood cancer, mesothelioma, myeloma, a myelodysplastic syndrome, lymphoma (e.g., non-Hodgkin's lymphoma, Hodgkin's lymphoma, and cutaneous T-cell lymphoma), leukemia (e.g., chronic myeloid leukemia, acute myelogenous leukemia, chronic lymphocytic leukemia, etc.), thymus cancer, etc.), a cutaneous system tumor (e.g., skin cancer, epidermoid cancer, melanoma, etc.), etc., as well as rhabdomyosarcoma, head and neck cancer, etc.

The term “subject” as described herein may be a human or non-human mammal, and the non-human mammal may be a wild animal, a zoo animal, an economic animal, a companion animal, a laboratory animal, etc. Preferably, the non-human mammal includes, but is not limited to, a pig, a cow, a sheep, a horse, a donkey, a fox, a raccoon dog, a mink, a camel, a dog, a cat, a rabbit, a rodent (e.g., a rat, a mouse, a guinea pig, a hamster, a gerbil, a chinchilla, and a squirrel), a monkey, etc.

The term “treating” as described herein refers to slowing, interrupting, arresting, controlling, stopping, reducing, or reversing the progression or severity of a sign, symptom, disorder, condition, or disease after the disease has begun to progress, but it does not necessarily involve the complete elimination of all disease-related signs, symptoms, conditions, or disorders.

The term “preventing” as described herein refers to the practice of preventing or delaying the onset of a disease or condition or symptom in the body.

The term “effective amount” as described herein refers to an amount or dose of the product of the present disclosure that provides a desired effect (e.g., treating, preventing, or inhibiting JAK1 expression) after administration to a subject or a cell or an organ thereof in single or multiple doses.

The term “lipid” as described herein refers to a group of organic compounds that include, but are not limited to, esters of fatty acids and are generally characterized by being poorly soluble in water, but soluble in many organic solvents.

The term “cationic lipid” as described herein refers to a lipid molecule capable of being positively charged.

The term “neutral lipid” as described herein refers to an uncharged non-phosphoglyceride lipid molecule.

The term “polyethylene glycol lipid” as described herein refers to a molecule comprising a lipid portion and a polyethylene glycol portion.

The term “lipid nanoparticle” as described herein refers to a particle with at least one dimension in the nanometer scale, which comprises at least one lipid.

The term “delivery system” as described herein refers to a formulation or composition that regulates the spatial, temporal, and dose distribution of a bioactive ingredient in an organism.

### BRIEF DESCRIPTION OF THE DRAWINGS

Hereinafter, examples of the present disclosure will be described in detail with reference to the accompanying drawings.

FIG. 1 shows the mass spectra of the sense strand (ss) and the antisense strand (as) of siJAK1-474.

FIG. 2 shows the mass spectra of the sense strand (ss) and the antisense strand (as) of siJAK1-826.

FIG. 3 shows the mass spectra of the sense strand (ss) and the antisense strand (as) of siJAK1-1131.

FIG. 4 shows the mass spectra of the sense strand (ss) and the antisense strand (as) of siJAK1-2200.

FIG. 5 shows the mass spectra of the sense strand (ss) and the antisense strand (as) of siJAK1-2714.

FIG. 6 shows the mass spectra of the sense strand (ss) and the antisense strand (as) of siJAK1-3283.

FIG. 7 shows the mass spectra of the sense strand (ss) and the antisense strand (as) of siJAK1-3630.

FIG. 8 shows the mass spectra of the sense strand (ss) and the antisense strand (as) of siJAK1-4096.

FIG. 9 shows the mass spectra of the sense strand (ss) and the antisense strand (as) of siJAK1-4359.

FIG. 10 shows the mass spectra of the sense strand (ss) and the antisense strand (as) of siJAK1-4389.

FIG. 11 shows the mass spectra of the sense strand (ss) and the antisense strand (as) of siJAK1-4410.

FIG. 12 shows the mass spectra of the sense strand (ss) and the antisense strand (as) of siJAK1-4627.

FIG. 13 shows the mass spectra of the sense strand (ss) and the antisense strand (as) of siJAK1-5026.

FIG. 14 shows the mass spectra of the sense strand (ss) and the antisense strand (as) of siJAK1-5034.

FIG. 15 shows the inhibitory effect of siJAK1 on IFN-γ-induced pSTAT in human keratinocytes.

FIG. 16 shows the inhibitory effect of siJAK1 on IFN-γ-induced pSTATE in mouse fibroblasts.

FIG. 17 shows the inhibitory effect of a modified siJAK sequence on IFN-γ-induced pSTAT1 in mouse fibroblasts.

### DETAILED DESCRIPTION

The technical solutions in the examples of the present disclosure will be described clearly and completely below with reference to the drawings. It is obvious that the described examples are only a part of the examples of the present disclosure, rather than all of them. Based on the examples of the present disclosure, all other examples obtained by those of ordinary skill in the art without creative work shall fall within the protection scope of the present disclosure.

### Example 1. siRNA Design and Synthesis

siRNAs were designed based on the transcript NM001321853.2 of human JAKC, and the target sequences and siRNA sequences are shown in Table 1.

TABLE 1
Target sequences and siRNA sequences of JAK1

| Name | Target sequence | Sense strand 5′-3′ | Antisense strand 5′-3′ |
|---|---|---|---|
| siJAK1- | GAATAAATGCAGTATCTAA | GAAUAAAUGCAGUAUCUA | UUAGAUACUGCAUUUAUU |
| 474 | (SEQ ID NO: 1) | A (SEQ ID NO: 15)dTdT | C (SEQ ID NO: 29)dTdT |
| siJAK1- | GGCATGGAACCAACGACAA | GGCAUGGAACCAACGACA | UUGUCGUUGGUUCCAUGC |
| 826 | (SEQ ID NO: 2) | A (SEQ ID NO: 16)dTdT | C (SEQ ID NO: 30)dTdT |
| siJAK1- | AAGCGATATATTCCAGAAAC | GCGAUAUAUUCCAGAAAC | UGUUUCUGGAAUAUAUCG |
| 1131 | ATT (SEQ ID NO: 3) | AUU (SEQ ID NO: 17) | CUU (SEQ ID NO: 31) |
| siJAK1- | GTTTCGATCGGATCCTCAA | GUUUCGAUCGGAUCCUCA | UUGAGGAUCCGAUCGAAA |
| 2200 | (SEQ ID NO: 4) | A (SEQ ID NO: 18)dTdT | C (SEQ ID NO: 32)dTdT |
| siJAK1- | GGTGCTGTCTAGGCAAGAA | GGUGCUGUCUAGGCAAGA | UUCUUGCCUAGACAGCAC |
| 2714 | (SEQ ID NO: 5) | A (SEQ ID NO: 19)dTdT | C (SEQ ID NO: 33)dTdT |
| siJAK1- | AGAACATTGTGAAGTACAA | AGAACAUUGUGAAGUACA | UUGUACUUCACAAUGUUC |
| 3283 | (SEQ ID NO: 6) | A (SEQ ID NO: 20)dTdT | U (SEQ ID NO: 34)dTdT |
| siJAK1- | GAATGTTTAATGCAATCTA | GAAUGUUUAAUGCAAUCU | UAGAUUGCAUUAAACAUU |
| 3630 | (SEQ ID NO: 7) | A (SEQ ID NO: 21)dTdT | C (SEQ ID NO: 35)dTdT |
| siJAK1- | GGCACACTGTAGTGCTTAA | GGCACACUGUAGUGCUUA | UUAAGCACUACAGUGUGC |
| 4096 | (SEQ ID NO: 8) | A (SEQ ID NO: 22)dTdT | C (SEQ ID NO: 36)dTdT |
| siJAK1- | GGCACATTAATCATAGATAA | CACAUUAAUCAUAGAUAA | UUUAUCUAUGAUUAAUGU |
| 4359 | AGA (SEQ ID NO: 9) | AGA (SEQ ID NO: 23) | GCC (SEQ ID NO: 37) |
| siJAK1- | ATGGACTTAGCCCTCAAATT | GGACUUAGCCCUCAAAUU | AAAUUUGAGGGCUAAGUC |
| 4389 | TCA (SEQ ID NO: 10) | UCA (SEQ ID NO: 24) | CAU (SEQ ID NO: 38) |
| siJAK1- | CAGTATCTATACAGTACTA | CAGUAUCUAUACAGUACU | UAGUACUGUAUAGAUACU |
| 4410 | (SEQ ID NO: 11) | A (SEQ ID NO: 25)dTdT | G (SEQ ID NO: 39)dTdT |
| siJAK1- | GCATGTTAATTCTCAAGAA | GCAUGUUAAUUCUCAAGA | UUCUUGAGAAUUAACAUG |
| 4627 | (SEQ ID NO: 12) | A (SEQ ID NO: 26)dTdT | C (SEQ ID NO: 40)dTdT |
| siJAK1- | GATTCAATGTGAACGATTA | GAUUCAAUGUGAACGAUU | UAAUCGUUCACAUUGAAU |
| 5026 | (SEQ ID NO: 13) | A (SEQ ID NO: 27)dTdT | C (SEQ ID NO: 41)dTdT |
| siJAK1- | GTGAACGATTACGGTCTTTA | GAACGAUUACGGUCUUUA | AUAAAGACCGUAAUCGUU |
| 5034 | TGA (SEQ ID NO: 14) | UGA (SEQ ID NO: 28) | CAC (SEQ ID NO: 42) |

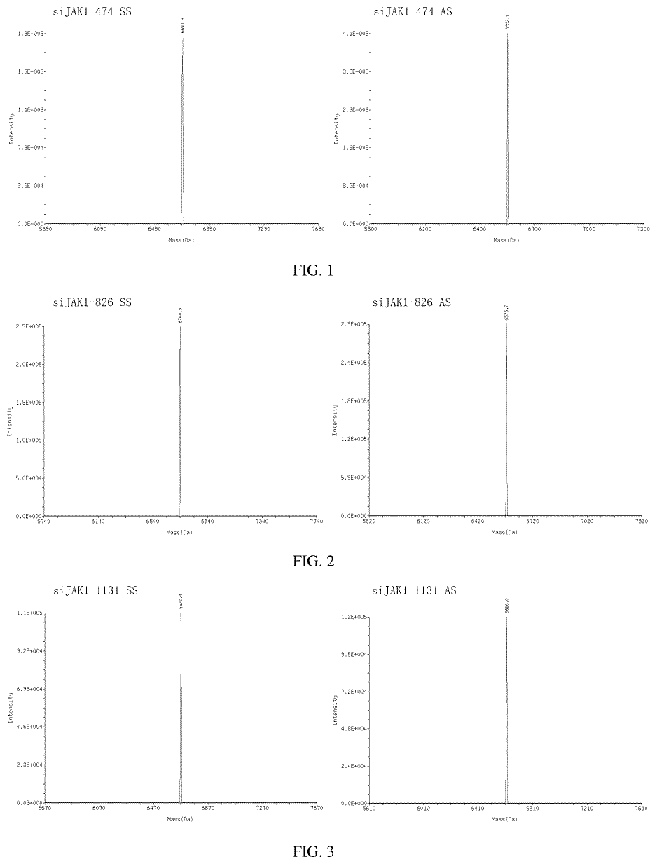

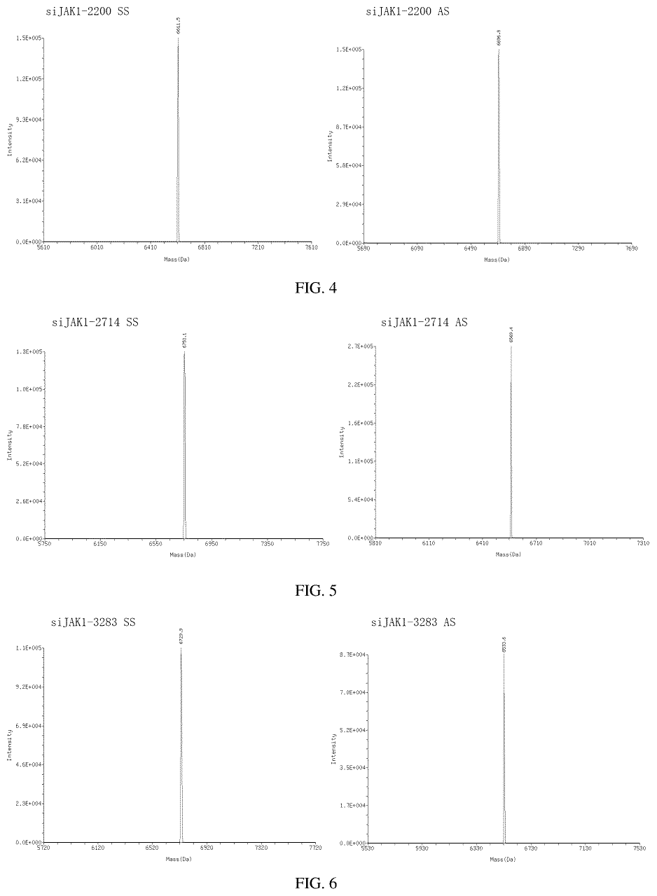

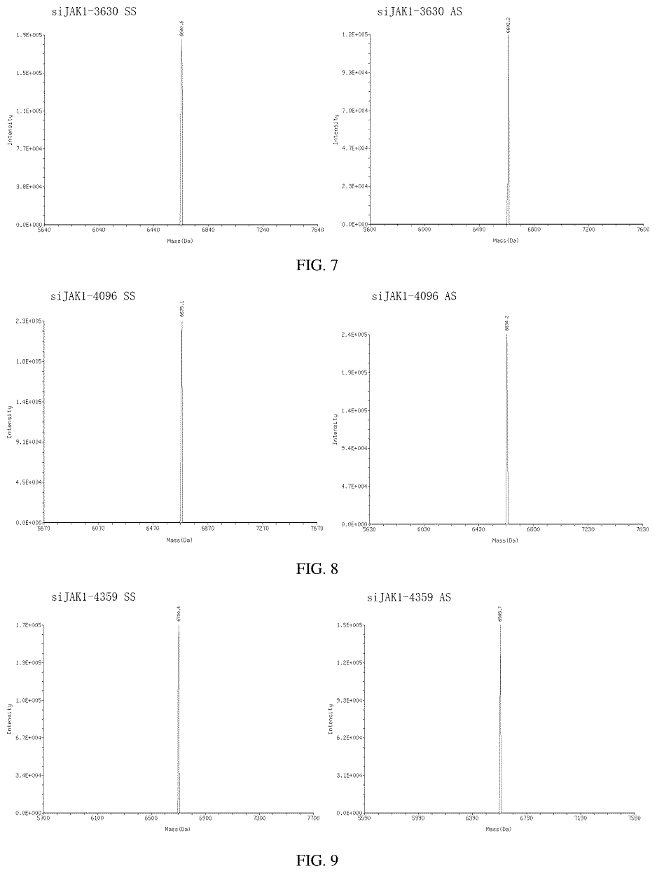

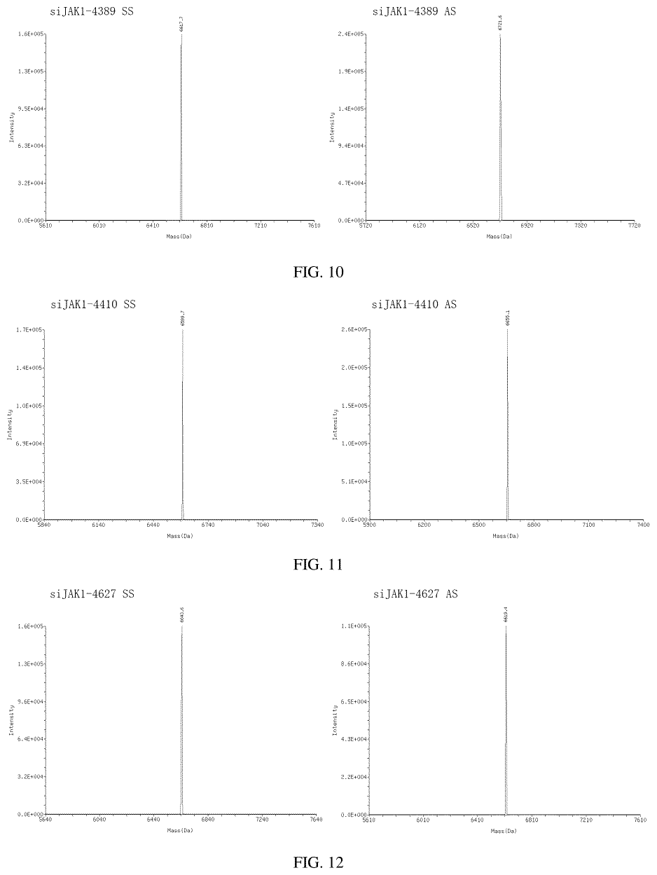

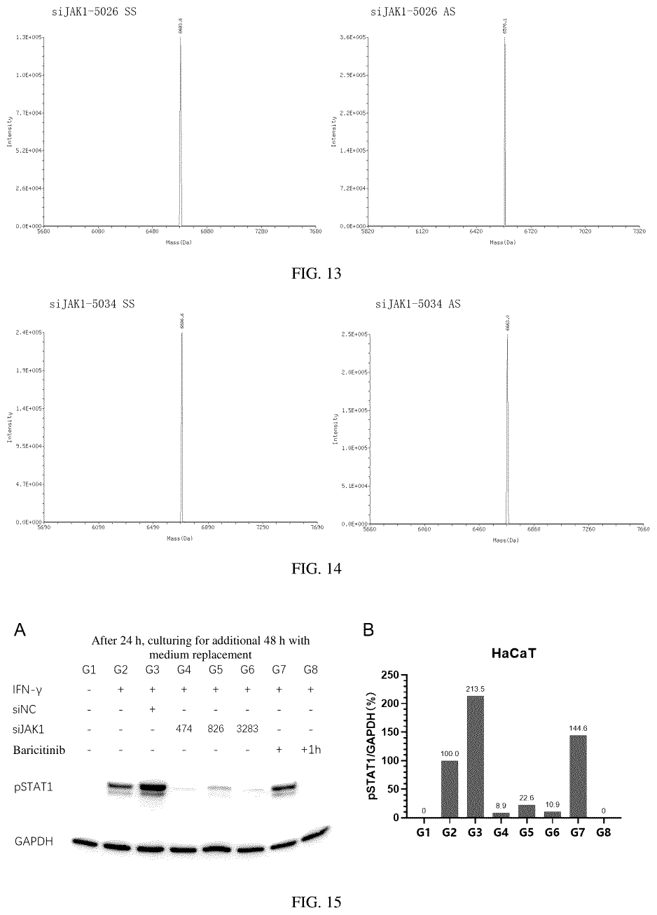

The siRNA sequences described above were synthesized by Beijing Tsingke Biotech Co., Ltd. A non-targeting control sequence (siNC, sense strand: 5′-UUC UCC GAA CGU GUC ACG UTT (SEQ ID NO: 43), antisense strand: 5′-ACG UGA CAC GUU CGG AGA ATT (SEQ ID NO: 44)) was designed and synthesized by Sangon Biotech (Shanghai) Co., Ltd. The mass spectra of the siRNAs shown in Table 1 are shown in FIGS. 1-14.

### Example 2: siRNA Screening by In Vitro Cell Experiment

### 1. Cell Transfection

One day prior to transfection, 293T cells were seeded into a 24-well cell culture plate at 1×10⁵ cells/well, and the culture medium was 500 μL of DMEM containing 10% FBS and 1% penicillin-streptomycin. Three replicate wells were made for each group. The culture plate was placed in an incubator with 5% CO₂ at 37° C. for 24 h to allow the cells to adhere to the wall. On the day of transfection, the culture medium was changed to 450 μL of DMEM culture medium without antibiotics (containing 10% FBS). The siRNAs were diluted with 25 μL of Opti-MEM culture medium, and the control group was siNC at the same concentration; a Lipofectamine™RNAiMAX transfection reagent (Lip) was diluted with another 25 μL of Opti-MEM culture medium, and the mixture was left to stand at room temperature for 5 min; and the two dilutions were uniformly mixed and left to stand at room temperature for 5 min. 50 μL of the mixed solution described above was added to each well to obtain a final transfection volume of 500 μL, and the working concentration of the siRNA was 50 nM. After the cells were further cultured in an incubator with 5% CO₂ at 37° C. for 48 h, the total RNA of the cells was extracted.

### 2. RNA Extraction

RNA was extracted using Eastep™ Super total RNA extraction kit, with the detailed procedures as follows:

1) 300 μL of RNA lysis solution and 300 μL of RNA diluent were added into each well of the 24-well cell culture plate, and the mixture was uniformly mixed and subsequently transferred to a 1.5 mL centrifuge tube. The mixture was centrifuged at 14000 g for 10 min, and the supernatant was carefully pipetted into a new 1.5 mL centrifuge tube.

2) A 0.5-fold volume of absolute ethanol was added, and the mixture was uniformly mixed by allowing the tube to be repeatedly upside down 15-20 times. Subsequently, the mixture was transferred to a centrifuge column and centrifuged at 12000 g for 1 min. The filtrate was discarded.

3) 600 μL of RNA wash buffer was added, and the mixture was centrifuged at 12000 g for 1 min. The filtrate was discarded.

4) 50 μL of DNase I incubation buffer was added, followed by DNA digestion at room temperature for 15 min.

5) 600 μL of RNA wash buffer was added, the mixture was centrifuged at 12000 g for 1 min, and the filtrate was discarded. This procedure was repeated once. Subsequently, the mixture was centrifuged at 14000 g for 2 min to remove the residual RNA wash buffer, and then the centrifuge column was placed in a collection tube.

6) A proper amount of RNase-free H₂O was added, and the mixture was left to stand for 15 min at room temperature, followed by centrifugation at 14000 g for 1 min. The RNA quality and concentration were detected using NanoDrop™. The extracted RNA was incubated at 70° C. for 5 min and immediately placed in an ice bath for 5 min for later use.

### 3. Detection of Gene Expression Level

1) The RNA was reverse-transcribed into a cDNA template using the All-in-One First-Strand Synthesis MasterMix (LABLEAD, Cat. No.: F0202) kit. The reaction system was established according to Table 2, where all components were mixed uniformly and centrifuged to collect the liquid at the bottom of a tube, and the PCR program was run according to the settings of 37° C. for 2 min, 55° C. for 15 min, and 85° C. for 5 min, thereby giving the cDNA template.

TABLE 2
Reverse transcription reaction system

| Reagent | Amount of use |
|---|---|
| RNA | 1 μg |
| All-in-One First-Strand Synthesis MasterMix | 4 μL |
| dsDNase | 1 μL |
| RNase-free H₂O | Supplement to 20 μL |

2) The gene expression level was detected using the Taq SYBR® Green qPCR Premix (LABLEAD, Cat. No.: R0202) kit, and the qPCR system was established according to Table 3. All components were mixed uniformly and centrifuged to collect the liquid at the bottom of a tube, and the PCR program was run according to the following settings:
Step1: 95° C. 30 s;
Step2: 95° C. 10 s;
Step3: 60° C. 30 s+Plate Read;
Step4: Go to Step2, 40 times;
Step5: Melt Curve 65 to 95° C., increment 0.5° C., for 5 s+Plate Read.

TABLE 3
qPCR reaction system

| Reagent | Amount of addition | |
|---|---|---|
| Taq SYBR ® Green qPCR Premix | 10 | μL |
| Forward primer (10 μM) | 0.4 | μL |
| Reverse primer (10 μM) | 0.4 | μL |
| cDNA template | 100 | ng |

| | |
|---|---|
| RNase-free H₂O | Supplement to 20 μL |

GAPDH was used as an endogenous reference to detect the relative expression level of JAK1 gene, and the sequences of the primers used are as follows:

GAPDH-F:
(SEQ ID NO: 45)
TCTGACTTCAACAGCGACAC;
GAPDH-R:
(SEQ ID NO: 46)
GCCAAATTCGTTGTCATACC;
JAK1-F:
(SEQ ID NO: 47)
AACCTCTTTGCCCTGTATGAC;
JAK1-R:
(SEQ ID NO: 48)
CTGCTCATTGTCGTTGGTTC.

The results demonstrated that in the 293T cells cultured for 48 h after siRNA transfection, the siRNAs targeting JAK1 designed in Example 1 were all capable of inhibiting gene expression compared to siNC (Table 4).

TABLE 4
Detection of the inhibitory effect of siJAK1 on the gene in the cells

Relative expression level of JAK1

| 293T | Mean value (Mean) | Standard deviation (SD) |
|---|---|---|
| siJAK1-474 | 20.24 | 1.17 |
| siJAK1-826 | 14.18 | 1.75 |
| siJAK1-1131 | 23.55 | 0.66 |
| siJAK1-2200 | 34.77 | 2.18 |
| siJAK1-2714 | 19.12 | 2.18 |
| siJAK1-3283 | 16.41 | 1.73 |
| siJAK1-3630 | 25.58 | 2.21 |
| siJAK1-4096 | 38.56 | 0.23 |
| siJAK1-4359 | 31.11 | 1.41 |
| siJAK1-4389 | 27.56 | 1.14 |
| siJAK1-4410 | 24.41 | 1.83 |
| siJAK1-4627 | 38.64 | 1.37 |
| siJAK1-5026 | 32.40 | 2.34 |
| siJAK1-5034 | 35.89 | 2.08 |
| siNC | 100.00 | 15.84 |

### Example 3: Inhibition of IFN-γ-induced STAT1 Phosphorylation by siJAK1 Transfection in Cells

One day prior to transfection, human keratinocyte HaCaT cells were seeded into a 6-well cell culture plate at 5×10⁵ cells/well, and the culture medium was 2 mL of MEM containing 15% FBS and 1% penicillin-streptomycin. Mouse fibroblast L929 cells were seeded into a 6-well cell culture plate at 1×10⁵ cells/well, and the culture medium was 2 mL of DMEM containing 10% FBS and 1% penicillin-streptomycin.

The grouping and treatment methods are shown in Table 5. On the day of transfection, the cells in Groups 3 to 6 were transfected with siNC, siJAK1-474, siJAK1-826, and siJAK1-3283, respectively, and the working concentration of all the siRNAs was 50 nM. Group 7 was treated with 5 μM baricitinib. After 24 h, the culture medium was replaced in all groups, and the cells were cultured for an additional 24-48 h. Prior to sample collection, 5 μM baricitinib was added in Group 8 and the cells were incubated in an incubator for 1 h. Subsequently, 100 ng/mL IFN-γ was added in Groups 2 to 8 and the cells were incubated in an incubator for 30 min. The cells were then harvested, and the total cellular proteins were extracted to detect the levels of phosphorylated STAT1 by Western blot.

TABLE 5
Grouping and treatment methods

| | G1 | G2 | G3 | G4 | G5 | G6 | G7 | G8 |
|---|---|---|---|---|---|---|---|---|
| IFN-γ 100 ng/ml | − | + | + | + | + | + | + | + |
| siNC 50 nM | − | − | + | − | − | − | − | − |
| siJAK1-474 50 nM | − | − | − | + | − | − | − | − |
| siJAK1-826 50 nM | − | − | − | − | + | − | − | − |
| siJAK-3283 50 nM | − | − | − | − | − | + | − | − |
| 5 μM of baricitinib | − | − | − | − | − | − | + | − |
| 5 μM of baricitinib (1 h) | − | − | − | − | − | − | − | + |

The protein extraction and Western blot procedures were as follows:

1) The cells were lysed in an appropriate volume of pre-chilled RIPA lysis solution (supplemented with 1 mM PMSF, R1090, LABLEAD prior to use; containing protease and phosphatase inhibitors) for 3-5 min. The lysates were centrifuged at 14000 g at 4° C. for 5 min. The supernatant was carefully pipetted into a new 1.5 mL centrifuge tube. Protein concentrations were measured using the BCA method (PC0020, Solarbio). The samples were mixed with a loading buffer and incubated in a water bath kettle at 95° C. for 10 min.

2) The protein samples were separated by electrophoresis on 4-12% 12-well gradient gels (P41212, LABLEAD) using compatible electrophoresis buffer. A total of 20 g of protein per lane was loaded, and a protein marker lane (P1018, LABLEAD) was set.

3) Membrane transfer: A PVDF membrane (Millipore, 0.45 m) of the same size as the gel and two thick filter papers were soaked in an appropriate amount of methanol until the membrane appeared translucent. The gel, membrane, and filter papers were subsequently soaked in a transfer buffer for 15 min. In a Bio-Red semi-dry transfer system, the filter paper, PVDF membrane, gel, and filter paper were placed in sequence. The proteins were transferred at 20 V constant voltage for 40 min to the PVDF membrane.

4) The PVDF membrane was trimmed based on the molecular weight of the protein and the marker, and blocked with 5% skim milk in TBST at room temperature for 1 h. Subsequently, the PVDF membrane was incubated overnight at 4° C. with primary antibodies against pSTAT1 (CST #9167) and GAPDH (GO100, LABLEAD), respectively. The next day, the membrane was washed three times in TBST for 5 min, followed by incubation with appropriate secondary antibodies for 1 h. After the membrane was washed again, chemiluminescent substrate (E1050, E1080, LABLEAD) was added, and signals were captured using the Bio-Red imaging system (ChemiDoc™ XRS+Imager) and analyzed using the accompanying software.

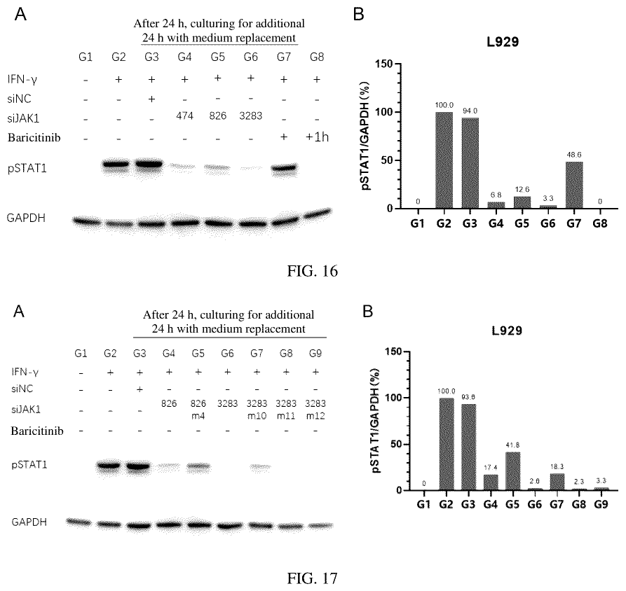

The results demonstrated that in the HaCaT cells, siJAK1 was capable of significantly inhibiting IFN-γ-induced STAT1 phosphorylation compared to Groups 2 and 3. Baricitinib, a marketed small-molecule inhibitor, significantly suppressed STAT1 phosphorylation after 1-hour treatment (Group 8), but its effect was almost completely lost after 72 hours of treatment (Group 7). In contrast, siJAK1 can still significantly inhibit STAT1 phosphorylation under the same treatment duration (FIG. 15). Similarly, in the L929 cells, siJAK1 can inhibit IFN-γ-induced STAT1 phosphorylation. After a single 48-hour treatment, the inhibitory effect of baricitinib was significantly reduced, while siJAK1 can still inhibit STAT1 phosphorylation (FIG. 16). The above results demonstrated that in in vitro experiments, siJAK1 not only inhibited the activation of the JAK/STAT signaling pathway, but also exhibited a longer-lasting effect than baricitinib, providing a foundation for the development of long-acting JAK inhibitors.

### Example 4: Inhibitory Effect of Modified siJAK1 Sequence on Gene Expression Level and IFN-γ-Induced STAT1 Phosphorylation

1. Design of Modified siJAK1 Sequences

The siJAK1-826 and 3283 sequences were chemically modified, and the modified sequences are shown in Table 6 below. m represents a 2′-OMe modification on the adjacent nucleotide to the right; f represents a 2′-F modification on the adjacent nucleotide to the right; Ψ represents that the adjacent nucleotide to the right is pseudouridine; and s represents a phosphorothioate modification on the phosphate backbone.

TABLE 6
Modified siJAK1 sequences

| Name | Sense strand 5′-3′ | Antisense strand 5′-3′ |
|---|---|---|
| JAK1-826- | GGCAΨGGAACCAACGACAAd | ΨUΨUGΨUCGΨUΨUGGΨUΨUCCAΨ |
| m4 | TdT (SEQ ID NO: 49) | UGCCdTdT (SEQ ID NO: 53) |
| JAK1-3283- | AGAACAΨUΨUGΨUGAAGΨUA | ΨUΨUGΨUACΨUΨUCACAAΨUGΨU |
| m10 | CAAdTdT (SEQ ID NO: 50) | ΨUCUdTdT (SEQ ID NO: 54) |
| JAK1-3283- | mAmGfAmAfCmAfUmUfGfUfGm | mUmUmGfUmAfCmUfUmCmAmCfA |
| m11 | AfAmGfUmAfCmAmAdTdT (SEQ | mAfUmGfUmUmCmUdTdT (SEQ ID |
| | ID NO: 51) | NO: 55) |
| JAK1-3283- | mAsmGsfAmAfCmAfUmUfGfUfG | mUsmUsmGfUmAfCmUfUmCmAmCf |
| m12 | mAfAmGfUmAfCsmAsmAdTdT | AmAfUmGfUmUsmCsmUdTdT (SEQ |
| | (SEQ ID NO: 52) | ID NO: 56) |

2. qPCR Analysis Ofinhibitory Effect on Gene Expression by Modified siJAK1 Sequences

According to the method described in Example 2, human keratinocyte HaCaT cells were transfected with the modified siJAK1 at an siRNA working concentration of 50 nM. The expression level of JAK1 was detected by qPCR 48 h after the transfection. The controls included siNC and unmodified siJAK1 at the same concentration. The results demonstrated that, compared to siNC, the modified siJAK1 was capable of significantly inhibiting the relative gene expression level (Table 7).

TABLE 7
Inhibitory effect of modified siJAK1 sequences on the gene

| | Relative expression level | |
|---|---|---|
| HaCaT | Mean value (Mean) | Standard deviation (SD) |
| siJAK1-826-m4 | 13.55 | 2.90 |
| siJAK1-3283-m10 | 15.89 | 0.67 |
| siJAK1-3283-m11 | 18.37 | 2.04 |
| siJAK1-3283-m12 | 19.20 | 1.58 |
| siJAK1-826 | 13.33 | 0.46 |
| siJAK1-3283 | 14.73 | 1.66 |
| siNC | 100.00 | 8.01 |

3. WB Detection of Inhibitory Effect of Modified siJAK1 Sequences on IFN-γ-Induced STAT1 Phosphorylation

According to the method described in Example 3, mouse fibroblast L929 cells were transfected with the modified siJAK1. The grouping and treatment methods are shown in Table 8. On the day of transfection, the cells in Groups 3 to 9 were transfected with siNC, siJAK1-826, siJAK1-826-m4, siJAK1-3283, siJAK1-3283-m10, siJAK1-3283-m11, and siJAK1-3283-m12, respectively, and the working concentration of all the siRNAs was 50 nM. After 24 h, the culture medium was replaced in all groups, and the cells were cultured for an additional 24 h. Prior to sample collection, 100 ng/mL IFN-γ was added in Groups 2 to 9 and the cells were incubated in an incubator for 30 min. The cells were then harvested, and the total cellular proteins were extracted to detect the levels of phosphorylated STAT1 by Western blot.

TABLE 8
Grouping and treatment methods

| | G1 | G2 | G3 | G4 | G5 | G6 | G7 | G8 | G9 |
|---|---|---|---|---|---|---|---|---|---|
| IFN-γ 100 ng/ml | − | + | + | + | + | + | + | + | + |
| siNC 50 nM | − | − | + | − | − | − | − | − | − |
| siJAK1-826 50 nM | − | − | − | + | − | − | − | − | − |
| siJAK1-826-m4 50 nM | − | − | − | − | + | − | − | − | − |
| siJAK-3283 50 nM | − | − | − | − | − | + | − | − | − |
| siJAK-3283-m10 50 nM | − | − | − | − | − | − | + | − | − |
| siJAK-3283-m11 50 nM | − | − | − | − | − | − | − | + | − |
| siJAK-3283-m12 50 nM | − | − | − | − | − | − | − | − | + |

The results demonstrated that, compared to siNC in Group 3, all the modified siJAK1 sequences were capable of inhibiting IFN-γ-induced STAT1 phosphorylation level; among them, the modified sequences siJAK1-3283-m11 and siJAK1-3283-m12 exhibited superior inhibitory effects (FIG. 17).

The preferred embodiments of the present disclosure are described in detail above, which, however, are not intended to limit the present disclosure. Within the scope of the technical concept of the present disclosure, various simple modifications can be made to the technical solution of the present disclosure, all of which will fall within the protection scope of the present disclosure.

In addition, it should be noted that the various specific technical features described in the above specific embodiments can be combined in any suitable manner in which the features do not contradict each other. In order to avoid unnecessary repetition, such combinations will not be illustrated separately in the present disclosure.

### The Partial Sequences Involved in the Present Disclosure are as Follows:

| Name | Target sequence | Sense strand 5′-3′ | Antisense strand 5′-3′ |
|---|---|---|---|
| siJAK1- | GAATAAATGCAGTAT | GAAUAAAUGCAGUAU | UUAGAUACUGCAUUU |
| 474 | CTAA (SEQ ID NO: 1) | CUAA (SEQ ID NO: | AUUC (SEQ ID NO: |
| | | 15)dTdT | 29)dTdT |
| siJAK1- | GGCATGGAACCAACG | GGCAUGGAACCAACG | UUGUCGUUGGUUCCA |
| 826 | ACAA (SEQ ID NO: 2) | ACAA (SEQ ID NO: | UGCC (SEQ ID NO: |
| | | 16)dTdT | 30)dTdT |
| siJAK1- | AAGCGATATATTCCA | GCGAUAUAUUCCAGA | UGUUUCUGGAAUAUA |
| 1131 | GAAACATT (SEQ ID | AACAUU (SEQ ID NO: | UCGCUU (SEQ ID NO: |
| | NO: 3) | 17) | 31) |
| siJAK1- | GTTTCGATCGGATCCT | GUUUCGAUCGGAUCC | UUGAGGAUCCGAUCG |
| 2200 | CAA (SEQ ID NO: 4) | UCAA (SEQ ID NO: | AAAC (SEQ ID NO: |
| | | 18)dTdT | 32)dTdT |
| siJAK1- | GGTGCTGTCTAGGCA | GGUGCUGUCUAGGCA | UUCUUGCCUAGACAG |
| 2714 | AGAA (SEQ ID NO: 5) | AGAA (SEQ ID NO: | CACC (SEQ ID NO: |
| | | 19)dTdT | 33)dTdT |
| siJAK1- | AGAACATTGTGAAGT | AGAACAUUGUGAAGU | UUGUACUUCACAAUG |
| 3283 | ACAA (SEQ ID NO: 6) | ACAA (SEQ ID NO: | UUCU (SEQ ID NO: |
| | | 20)dTdT | 34)dTdT |
| siJAK1- | GAATGTTTAATGCAA | GAAUGUUUAAUGCAA | UAGAUUGCAUUAAAC |
| 3630 | TCTA (SEQ ID NO: 7) | UCUA (SEQ ID NO: | AUUC (SEQ ID NO: |
| | | 21)dTdT | 35)dTdT |
| siJAK1- | GGCACACTGTAGTGC | GGCACACUGUAGUGC | UUAAGCACUACAGUG |
| 4096 | TTAA (SEQ ID NO: 8) | UUAA (SEQ ID NO: | UGCC (SEQ ID NO: |
| | | 22)dTdT | 36)dTdT |
| siJAK1- | GGCACATTAATCATA | CACAUUAAUCAUAGA | UUUAUCUAUGAUUAA |
| 4359 | GATAAAGA (SEQ ID | UAAAGA (SEQ ID NO: | UGUGCC (SEQ ID NO: |
| | NO: 9) | 23) | 37) |
| siJAK1- | ATGGACTTAGCCCTC | GGACUUAGCCCUCAA | AAAUUUGAGGGCUAA |
| 4389 | AAATTTCA (SEQ ID | AUUUCA (SEQ ID NO: | GUCCAU (SEQ ID NO: |
| | NO: 10) | 24) | 38) |
| siJAK1- | CAGTATCTATACAGT | CAGUAUCUAUACAGU | UAGUACUGUAUAGAU |
| 4410 | ACTA (SEQ ID NO: 11) | ACUA (SEQ ID NO: | ACUG (SEQ ID NO: |
| | | 25)dTdT | 39)dTdT |
| siJAK1- | GCATGTTAATTCTCAA | GCAUGUUAAUUCUCA | UUCUUGAGAAUUAAC |
| 4627 | GAA (SEQ ID NO: 12) | AGAA (SEQ ID NO: | AUGC (SEQ ID NO: |
| | | 26)dTdT | 40)dTdT |
| siJAK1- | GATTCAATGTGAACG | GAUUCAAUGUGAACG | UAAUCGUUCACAUUG |
| 5026 | ATTA (SEQ ID NO: 13) | AUUA (SEQ ID NO: | AAUC (SEQ ID NO: |
| | | 27)dTdT | 41)dTdT |
| siJAK1- | GTGAACGATTACGGT | GAACGAUUACGGUCU | AUAAAGACCGUAAUC |
| 5034 | CTTTATGA (SEQ ID | UUAUGA (SEQ ID NO: | GUUCAC (SEQ ID NO: |
| | NO: 14) | 28) | 42) |

SEQ ID NO: 43 non-targeting control sequence siNC, sense strand
5′-UUC UCC GAA CGU GUC ACG UTT
SEQ ID NO: 44 non-targeting control sequence siNC, antisense strand
5′-ACG UGA CAC GUU CGG AGA ATT
SEQ ID NO: 45 GAPDH-F
TCTGACTTCAACAGCGACAC
SEQ ID NO: 46 GAPDH-R
GCCAAATTCGTTGTCATACC
SEQ ID NO: 47 JAK1-F
AACCTCTTTGCCCTGTATGAC
SEQ ID NO: 48 JAK1-R
CTGCTCATTGTCGTTGGTTC
SEQ ID NO: 49 JAK1-826-m4 sense strand
GGCAUGGAACCAACGACAAdTdT
SEQ ID NO: 53 JAK1-826-m4 antisense strand
ΨUΨUGΨUCGΨUΨUGGΨUΨUCCAΨUGCCdTdT
SEQ ID NO: 50 JAK1-3283-m10 sense strand
AGAACAΨUΨUGΨUGAAGΨUACAAdTdT
SEQ ID NO: 54 JAK1-3283-m10 antisense strand
ΨUΨUGΨUACΨUΨUCACAAΨUGΨUΨUCΨUdTdT
SEQ ID NO: 51 JAK1-3283-m11 sense strand
mAmGfAmAfCmAfUmUfGfUfGmAfAmGfUmAfCmAmAdTdT
SEQ ID NO: 55 JAK1-3283-m11 antisense strand
mUmUmGfUmAfCmUfUmCmAmCfAmAfUmGfUmUmCmUdTdT
SEQ ID NO: 52 JAK1-3283-m12 sense strand
mAsmGsfAmAfCmAfUmUfGfUfGmAfAmGfUmAfCsmAsmAdTdT
SEQ ID NO: 56 JAK1-3283-m12 antisense strand
mUsmUsmGfUmAfCmUfUmCmAmCfAmAfUmGfUmUsmCsmUdTdT

1. An interfering RNA targeting JAK1, wherein a target site sequence of the interfering RNA comprises a nucleotide sequence set forth in any one or two or more of SEQ ID NOs: 1-14.

2. The interfering RNA according to claim 1, comprising one or a combination of two or more of an siRNA, a dsRNA, an shRNA, an aiRNA, or an miRNA.

3. The interfering RNA according to claim 1, further comprising an over-hang,
wherein the over-hang is located at the 3′ terminus of a sense strand and/or an antisense strand of the interfering RNA;
wherein the over-hang comprises, but is not limited to, dTdT, UU, or UG.

4. The interfering RNA according to claim 1, comprising a sense strand and/or an antisense strand,
the sense strand comprises a nucleotide sequence set forth in any one or two or more of SEQ ID NOs: 15-28;
the antisense strand comprises a nucleotide sequence set forth in any one or two or more of SEQ ID NOs: 29-42.

5. The interfering RNA according to claim 1, further comprising at least one modification, wherein the modification comprises a modification on a base, a sugar ring, and/or a phosphate backbone;
the modification on a base comprises, but is not limited to, a pyrimidine modification at position 5, a purine modification at position 8, a pseudouridine modification, and/or a 5-bromouracil substitution;
the modification on a sugar ring comprises, but is not limited to, a 2′-hydroxyl modification, a 2′-fluoro modification, a 2′-deoxy modification, a 2′-alkyl modification, a 2′-O-methyl modification, a 2′-O-methoxyethyl modification, a 2′-O-allyl modification, a 2′-C-allyl modification, and a locked nucleotide modification;
the modification on a phosphate backbone comprises, but is not limited to, a phosphorothioate modification and a ligand modification.

6. The interfering RNA according to claim 5, wherein the modification further comprises nucleotides having inosine, queuosine, xanthine, 2′-methylribose, a non-natural phosphodiester bond (e.g., methylphosphonate and phosphorothioate), and/or a peptide.

7. The interfering RNA according to claim 5, wherein the ligand is selected from any one or a combination of two or more of cholesterol, biotin, a vitamin, a galactose derivative or analog, a lactose derivative or analog, N-acetylgalactosamine (GalNAc), an N-acetylgalactosamine derivative or analog, an N-acetylglucosamine derivative or analog, and a mannose 6-phosphate (M6P) derivative or analog.

8. The interfering RNA according to claim 7, wherein the N-acetylgalactosamine derivative or analog has a structural formula as follows:

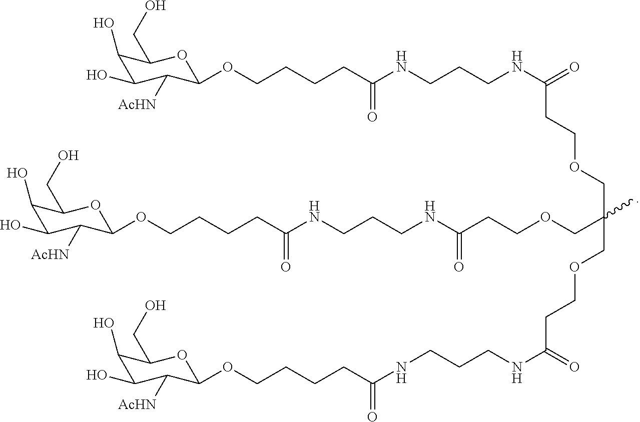 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

9. A delivery system for the interfering RNA according to claim 1, the delivery system comprising the interfering RNA according to claim 1 and a vector.

10. The delivery system according to claim 9, wherein the vector is a viral vector or a non-viral vector;
the viral vector comprises one or a combination of two or more of a lentivirus vector, a retrovirus vector, an adenovirus vector, an adeno-associated virus vector, a poxvirus vector, or a herpesvirus vector;
the non-viral vector comprises any one or a combination of two or more of a liposome, a lipid nanoparticle, a polymer, a polypeptide, an antibody, an aptamer, and N-acetylgalactosamine (GalNAc).

11. The delivery system according to claim 10, wherein the lipid nanoparticle/the liposome comprises one or a combination of two or more of a cationic lipid, a neutral lipid, a polyethylene glycol lipid, a steroidal lipid, and an anionic lipid.

12. The delivery system according to claim 11, wherein the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl)azanediyl]di(hexane-6,1-diyl) bis(2-hexyldecanoate) (ALC-0315), 1,2-dioleoyloxy-3-(trimethylammonium)propane (DOTAP), 1,2-di-(9Z-octadecenoyl)-3-trimethylammonium-propane and 1,2-dihexadecanoyl-3-trimethylammonium-propane, 3β-[N-(N′,N′-dimethylaminoethane)-carbamoyl]cholesterol (DC-cholesterol), dimethyldioctadecylammonium (DDA), 1,2-dimyristoyl-3-trimethylammonium propane (DMTAP), dipalmitoyl(C16:0)trimethylammonium propane (DPTAP), distearoyltrimethylammonium propane (DSTAP), N-[1-(2,3-diallyloxy)propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2(spermine-carboxamido)ethyl]-N,N-dimethyl-1-propanaminium trifluoroacetate (DOSPA), 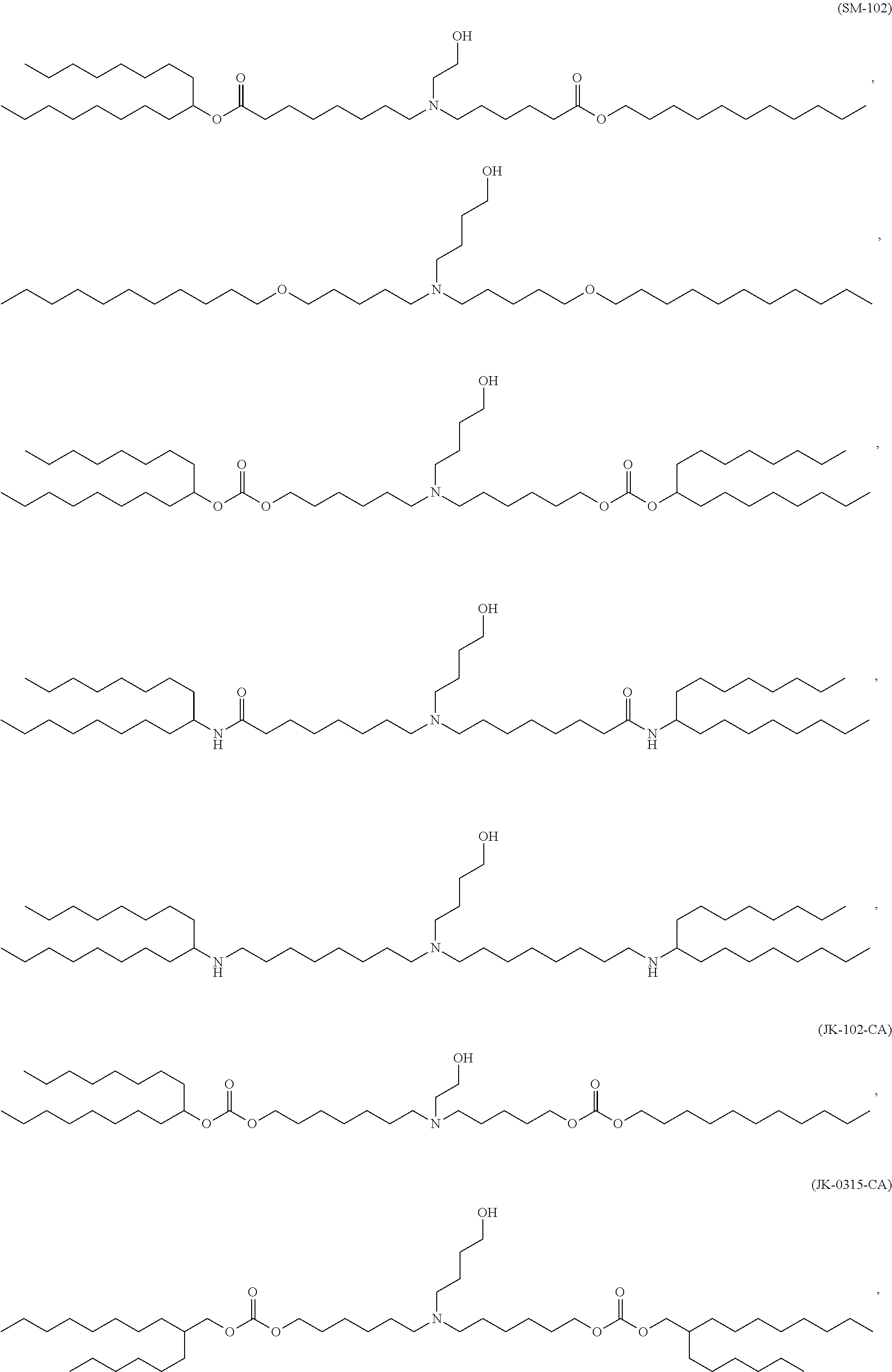
and dilinoleyl 4-(N,N-dimethylamino)butanoate (Dlin-MC3-DMA).

13. The delivery system according to claim 11, wherein the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), distearoylphosphatidylethanolamine (DSPE), etc.

14. The delivery system according to claim 11, wherein the polyethylene glycol lipid comprises one or a combination of two or more of 2-[(polyethylene glycol)-2000]—N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA), 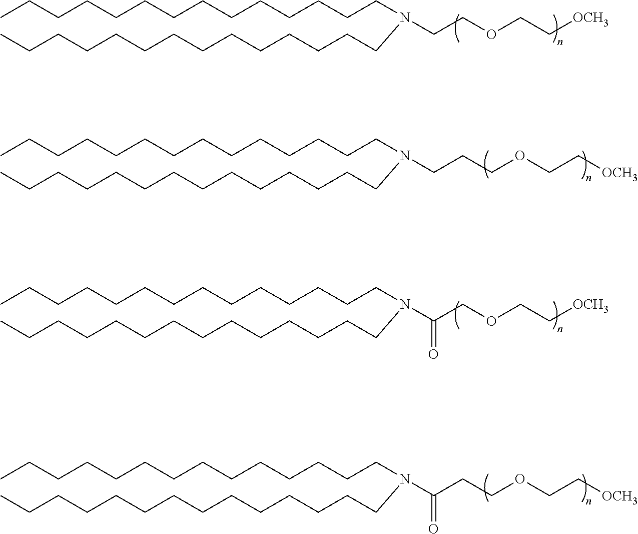
wherein n is an integer selected from 20-300.

15. The delivery system according to claim 11, wherein the polyethylene glycol lipid is a polyethylene glycol lipid with a single molecular weight, and, the polyethylene glycol lipid comprises:

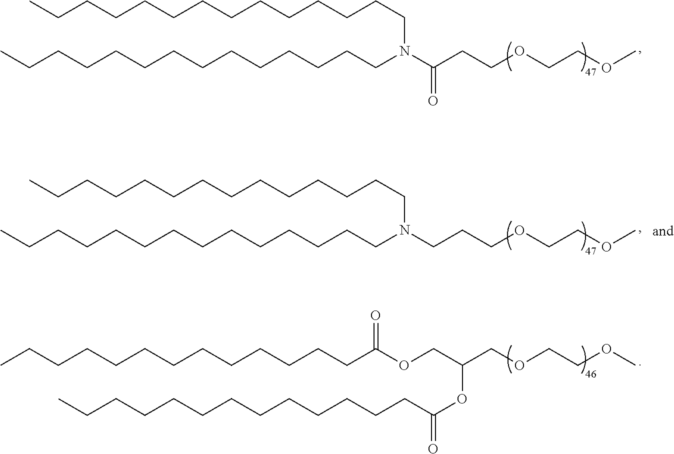

16. The delivery system according to claim 11, wherein the cationic lipid is a steroid-cationic lipid compound,
and the compound has a structure of:

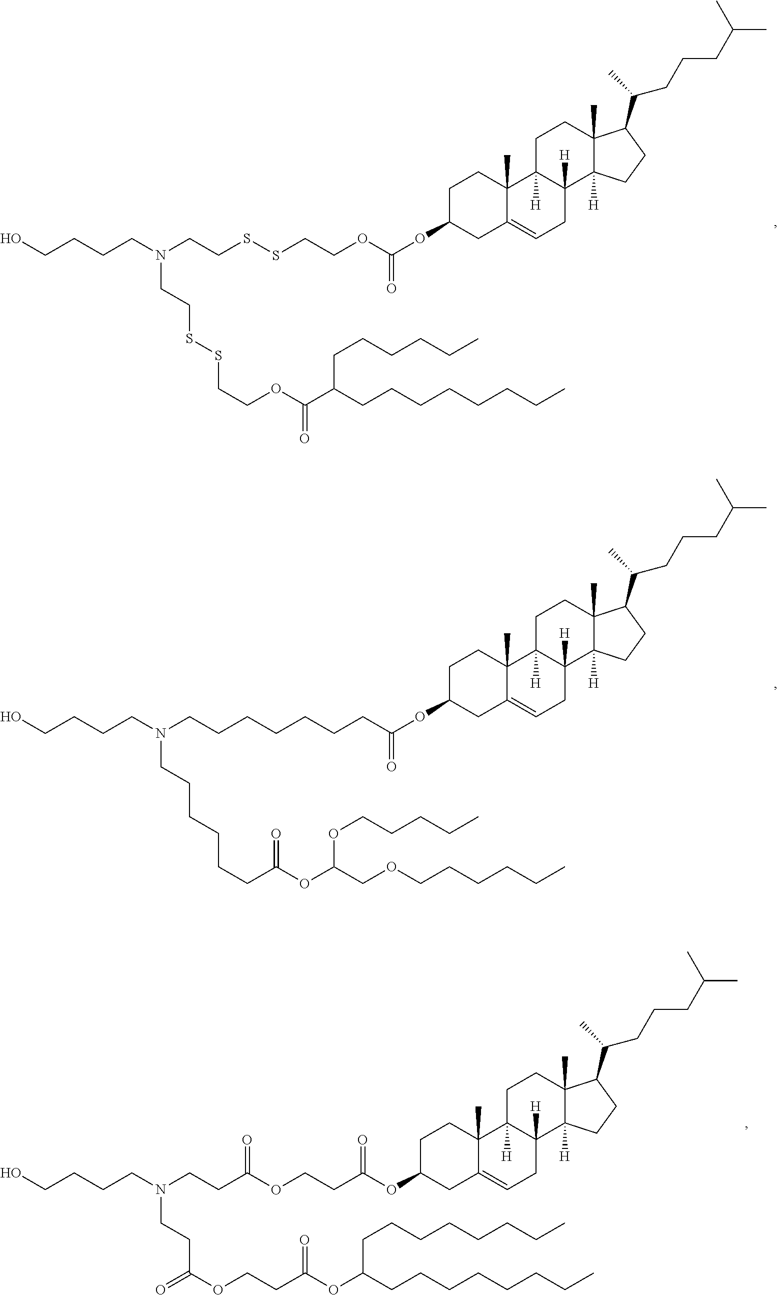

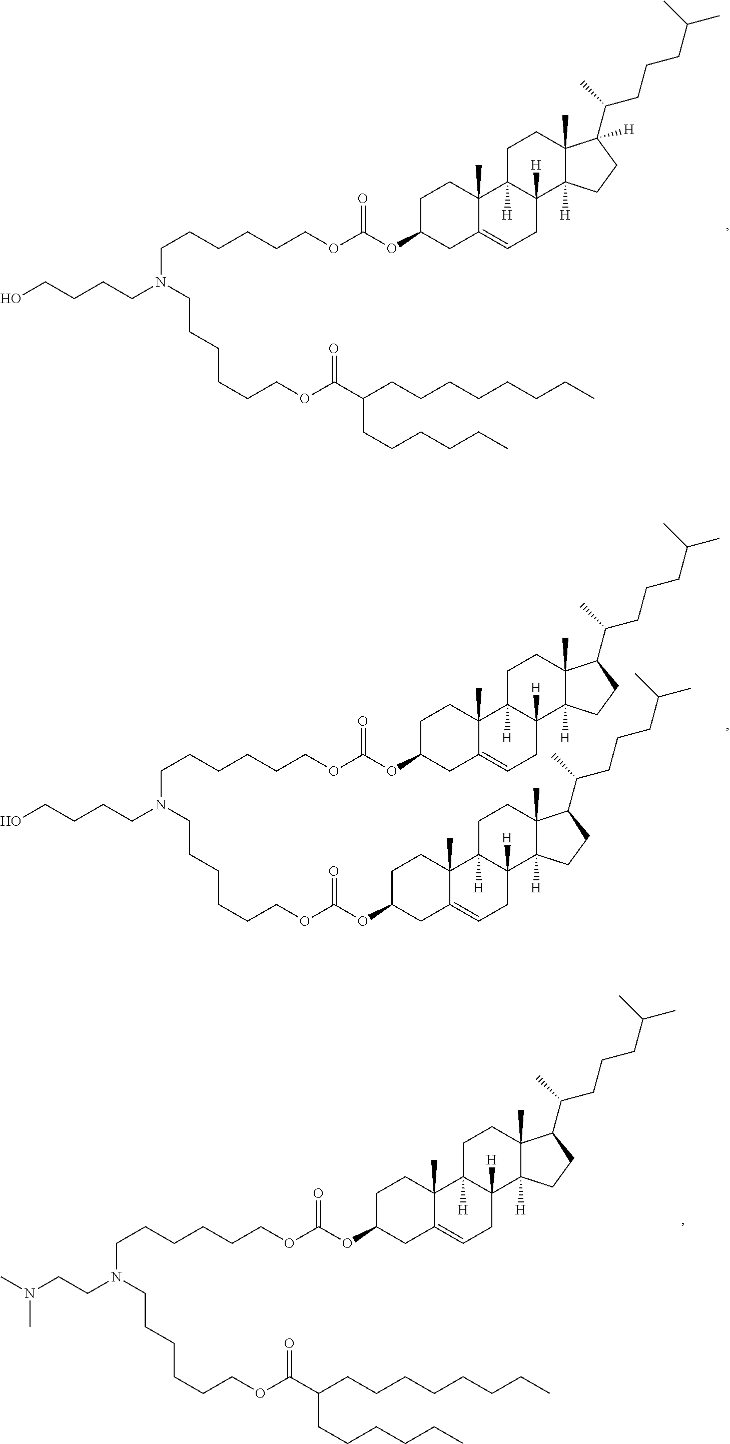

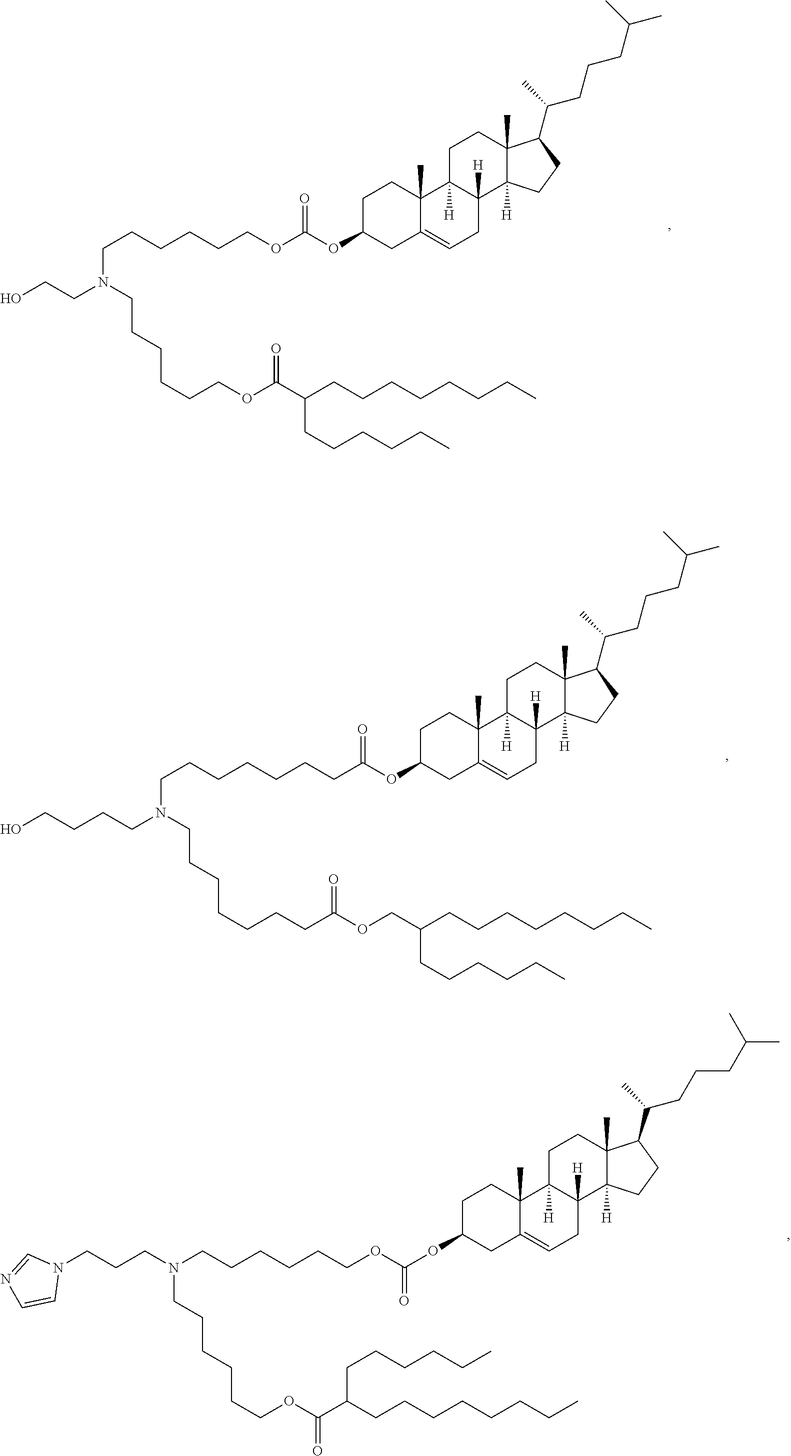

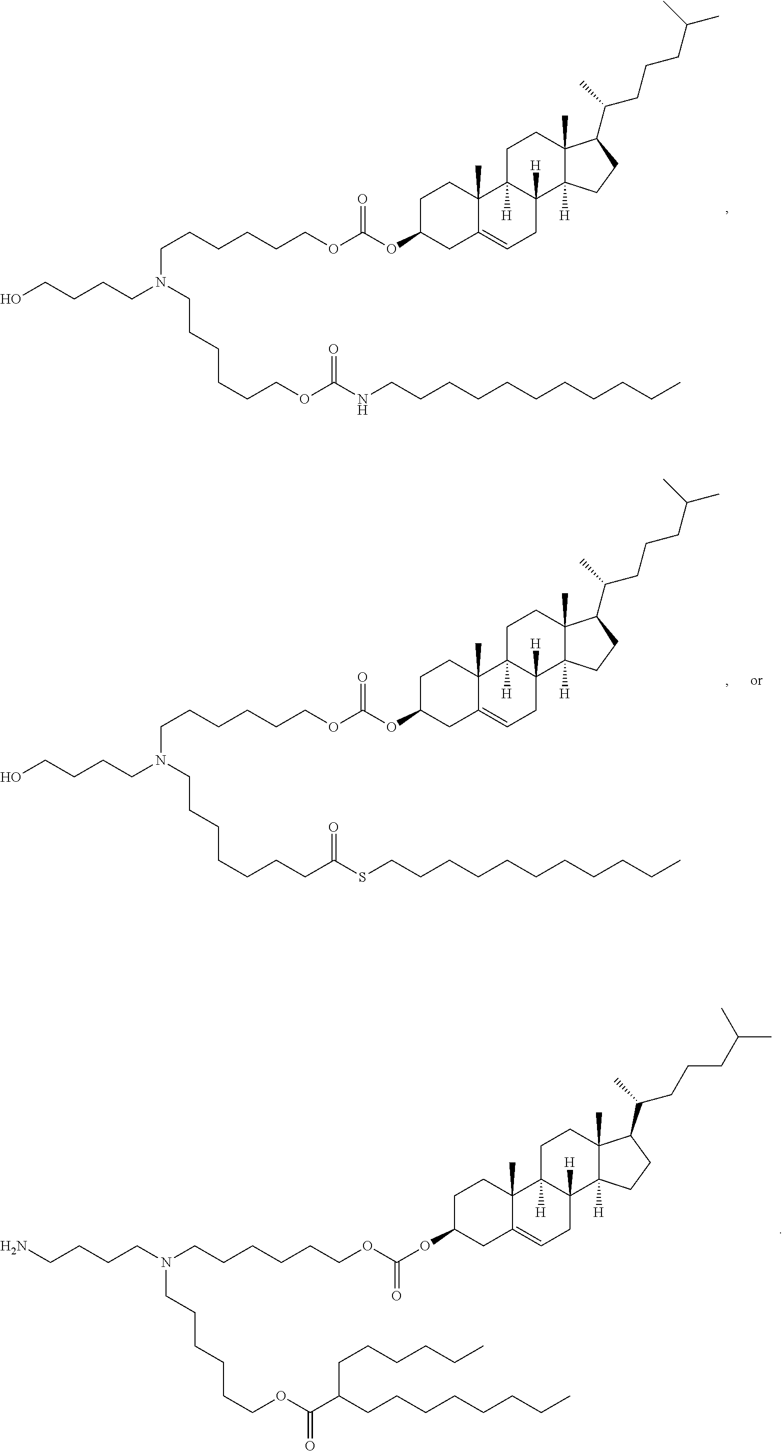

17. The delivery system according to claim 11, wherein the anionic liposome comprises one or a combination of two or more of dioleoyl phosphatidylglycerol, dioleoyl phosphatidylethanolamine, etc.

18. The delivery system according to claim 11, wherein the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrochole sterol, dihydroergo sterol, dinosterol, epicholesterol, ergosterol, fucosterol, hexahydrolumisterol, hydroxychole sterol, lanosterol, lumisterol, saringosterol, sitostanol, sitosterol, stigmastanol, stigmasterol, cholic acid, glycocholic acid, taurocholic acid, deoxycholic acid, and lithocholic acid.

19. A method for inhibiting JAK1 expression, comprising adding the interfering RNA according to claim 1, or the delivery system comprising the interfering RNA thereof.

20. A method for treating a JAK/STAT signaling pathway dysregulation-associated disease, comprising administering to a subject a therapeutically effective amount of the interfering RNA according to claim 1, or the delivery system comprising the interfering RNA thereof.
